(19) United States
(12) Patent Application Publication
(10) Pub. No.: US 2026/0018301 A1
(43) Pub. Date: Jan. 15, 2026

(54) MACHINE LEARNING DRIVEN IDENTIFICATION OF GENE-EXPRESSION SIGNATURES ASSOCIATED WITH PERSISTENT MULTIPLE ORGAN DYSFUNCTION

(71) Applicants: CHILDREN'S HOSPITAL MEDICAL CENTER, Cincinnati, OH (US); EMORY UNIVERSITY, Atlanta, GA (US)

(72) Inventors: Mihir ATREYA, Cincinnati, OH (US); Rishikesan KAMALESWARAN, Suwanee, GA (US); Shayantan BANERJEE, Kolkata (IN)

(73) Assignees: CHILDREN'S HOSPITAL MEDICAL CENTER, Cincinnati, OH (US); EMORY UNIVERSITY, Atlanta, GA (US)

(21) Appl. No.: 19/122,085

(22) PCT Filed: Oct. 27, 2023
(86) PCT No.: PCT/US2023/078012

§ 371 Date: Apr. 17, 2025

Related U.S. Application Data
(60) Provisional application No. 63/420,416, filed on Oct. 28, 2022.

(51) Int. Cl. G16H 50/30; C12Q 1/6883; G16B 25/10; G16B 40/20; G16H 10/60; G16H 50/70

(52) U.S. Cl. CPC G16H 50/30; C12Q 1/6883; G16B 25/10; G16B 40/20; G16H 10/60; G16H 50/70; C12Q 2600/158

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to septic shock in pediatric patients. Certain aspects of the disclosure relates to identifying one or more biomarkers associated with septic shock in pediatric patients in combination with one or more endothelial-derived biomarkers, obtaining a sample from a pediatric patient having at least one indication of septic shock, then quantifying from the sample an amount of said biomarkers, wherein the level of said biomarker correlates with a predicted outcome.

### CROSS REFERENCE TO RELATED APPLICATION

The present application claims the benefit of priority under 35 U.S.C. § 119(e) to U.S. Provisional Application No. U.S. Patent Application No. 63/420,416, MACHINE LEARNING DRIVEN IDENTIFICATION OF GENE-EXPRESSION SIGNATURES CORRELATED WITH MULTIPLE ORGAN DYSFUNCTION TRAJECTORIES AND COMPLEX SUB-ENDOTYPES OF PEDIATRIC SEPTIC SHOCK, filed on filed Oct. 28, 2022, which is currently co-pending herewith and which is incorporated by reference in its entirety.

### STATEMENT REGARDING FEDERALLY-SPONSORED RESEARCH

This invention was made with government support under Grant Nos. R35 GM126943, R21 GM151703, and R01 GM139967 awarded by the National Institutes of Health. The government has certain rights in the invention.

### FIELD

The disclosure herein generally relates to the identification and validation of clinically relevant, quantifiable biomarkers of diagnostic and therapeutic responses for blood, vascular, cardiac, and respiratory tract dysfunction, having particular utility as biomarkers associated with multiple organ dysfunction syndrome (MODS) and/or septic shock.

### BACKGROUND

Multiple organ dysfunction syndrome (MODS) disproportionately drives sepsis morbidity and mortality among critically ill patients. In particular, MODS is a major cause for mortality among children admitted to intensive care units [1]. Those who survive the acute phase remain at high risk of new morbidity, including technology dependence [2], nosocomial infections [3], and late death [4, 5]. Despite the significant burden of disease, care for patients with MODS remains limited to organ support, with no disease modifying therapies currently proven to improve clinical outcomes. Although numerous clinical phenotypes of sepsis-associated MODS have been described [3,6,7], there is a need for a systematic understanding of MODS pathobiology in order to develop approaches to identify at-risk patients.

Biologic heterogeneity in sepsis has hindered therapeutic development [35]. Precision medicine approaches offer promising solutions to address the underlying heterogeneity [36]. Predictive enrichment, which involves identification of patient subclasses based on shared biological pathways that may be amenable to intervention [37], is one such approach. Over a decade ago, Dr. Wong and colleagues, first identified patient subclasses through whole blood genome-wide expression profiling of children with septic shock [8,38]. Among ˜7,000 differentially regulated genes, 100 subclass defining genes, which corresponded to the adaptive immune system and glucocorticoid receptor signaling, were identified. Subsequently, exposure to adjuvant steroid therapy among endotype A patients was shown to be independently associated with an increased risk of sepsis mortality [15,39,40].

However, current endotyping strategies rely on either distinguishing the septic shock signature relative to patients with systemic inflammatory response syndrome (SIRS) and healthy controls [8,15,38] or unsupervised machine learning in which patients are grouped based on similarities across multiple dimensions of gene-expression data [9,10,12,14,34], and have largely focused on mortality as an outcome. Such strategies do not take into account the significant underlying heterogeneity associated with MODS.

### SUMMARY

Embodiments of the disclosure relate to methods of classifying a patient with septic shock as high risk of persistent multiple organ dysfunction syndrome (MODS) trajectory and/or mortality or other than high risk of persistent MODS trajectory and/or mortality, the method including: obtaining a sample from a pediatric patient with septic shock at a first time point; analyzing the sample to determine gene expression levels of two or more biomarkers selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8; determining whether the gene expression levels of each of the at least two biomarkers are differentially expressed normalized gene expression levels; and classifying the patient as high risk of persistent MODS trajectory and/or mortality, or other than high risk of persistent MODS trajectory and/or mortality, based on the determination of whether the expression levels of each of the at least two biomarkers are differentially expressed normalized gene expression levels.

In some embodiments, a classification of high risk of persistent MODS trajectory and/or mortality includes a differentially expressed normalized expression level of 3, 4, 5, 6, 7, 8, 9, 10, 11, 12, 13, 14, 15, 16, 17, 18, 19, 20, or more biomarkers selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8. In some embodiments, a classification of high risk of persistent MODS trajectory and/or mortality includes a non-differentially expressed normalized gene expression level of RUNX1.

In some embodiments, a classification of high risk of persistent MODS trajectory and/or mortality can include a differentially expressed normalized expression level of 3, 4, 5, 6, 7, 8, 9, 10, 11, 12, 13, 14, 15, 16, 17, 18, 19, or 20 biomarkers selected from: RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8. In some embodiments, a classification of high risk of persistent MODS trajectory and/or mortality can include a differentially expressed normalized expression level of 20 biomarkers including: RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8.

In some embodiments, a classification of high risk of persistent MODS trajectory and/or mortality can include a differentially expressed normalized gene expression level of 3, 4, 5, 6, 7, 8, 9, or 10 biomarkers selected from: RETN, ADAMTS3, LDHA, LCN2, IL1R2, DDIT4, CEACAM8, MERTK, MPO, and ARL4A. In some embodiments, a classification of high risk of persistent MODS trajectory and/or mortality can include differentially expressed normalized expression levels of all biomarkers selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8.

In some embodiments, determining whether the gene expression levels of each of the at least two biomarkers are differentially expressed normalized gene expression levels can include comparing the gene expression levels to respective gene expression levels from a normal, healthy subject. In some embodiments, the patient can be classified as high risk of persistent MODS trajectory and/or mortality, or other than persistent MODS trajectory and/or mortality, when the expression levels of each of the at least two biomarkers are differentially expressed normalized gene expression levels as compared to respective gene expression levels from a normal, healthy subject.

Some embodiments of the disclosure include methods of classifying a patient with septic shock as high risk of cardiovascular, respiratory, or renal dysfunction or other than high risk of cardiovascular, respiratory, or renal dysfunction, the method including: obtaining a sample from a pediatric patient with septic shock at a first time point; analyzing the sample to determine gene expression levels of two or more biomarkers selected from genes listed in Tables 13-24; determining whether the gene expression levels of each of the at least two biomarkers are differentially expressed normalized gene expression levels; and classifying the patient as high risk of cardiovascular, respiratory, or renal dysfunction, or other than high risk of cardiovascular, respiratory, or renal dysfunction, based on the determination of whether the expression levels of each of the at least two biomarkers are differentially expressed normalized gene expression levels.

In some embodiments, determining whether the gene expression levels of each of the at least two biomarkers are differentially expressed normalized gene expression levels includes comparing the gene expression levels to respective gene expression levels from a normal, healthy subject. In some embodiments, the patient can be classified as high risk of cardiovascular, respiratory, or renal dysfunction, or other than high risk of cardiovascular, respiratory, or renal dysfunction, when the expression levels of each of the at least two biomarkers are differentially expressed normalized gene expression levels as compared to respective gene expression levels from a normal, healthy subject.

In some embodiments, biomarker expression levels can be determined by quantification of serum biomarker concentrations. In some embodiments, gene expression levels can be determined by concentrations and/or by cycle threshold (CT) values.

In some embodiments, a classification other than high risk includes a classification of low risk or intermediate risk.

In some embodiments, MODS includes cardiovascular, respiratory, renal, hepatic, hematologic, and/or neurologic dysfunction. In some embodiments, MODS includes cardiovascular dysfunction. In some embodiments, MODS includes dysfunction in one or more organs selected from heart, lungs, kidneys, liver, blood, and brain. In some embodiments, high risk of persistent MODS trajectory and/or mortality by day 7 of septic shock or other than high risk of persistent MODS trajectory and/or mortality by day 7 of septic shock can be determined.

In some embodiments, the classification can be combined with one or more patient demographic data and/or clinical characteristics and/or results from other tests or indicia of septic shock and/or one or more additional biomarkers. In some embodiments, the one or more additional biomarkers can include C-C Chemokine ligand 3 (CCL3), C-C Chemokine ligand 4 (CCL4), Granzyme B (GZMB), Interleukin-1α (IL-1a), Matrix metallopeptidase 8 (MMP8), Angiopoietin-1 (Angpt-1), Angiopoietin-2 (Angpt-2), Tyrosine kinase with immunoglobulin-like loops and epidermal growth factor homology domains-2 (Tie-2), Vascular cell adhesion molecule-1 (VCAM-1), P-selectin, E-selectin, and/or Platelet and endothelial cell adhesion molecule-1 (PECAM-1). In some embodiments, the patient demographic data and/or clinical characteristics and/or results from other tests or indicia of septic shock can include at least one selected from: the septic shock causative organism, the presence or absence or chronic disease, and/or the age, gender, race, and/or co-morbidities of the patient.

In some embodiments, the classification can be combined with one or more additional population-based risk scores. In some embodiments, the one or more population-based risk scores can include Pediatric Sepsis Biomarker Risk Model (PERSEVERE), Pediatric Risk of Mortality (PRISM), PRISM III, Pediatric Index of Mortality (PIM), and/or Pediatric Logistic Organ Dysfunction (PELOD).

In some embodiments, the sample can be obtained within the first hour of presentation with septic shock. In some embodiments, the sample can be obtained within the first 24 hours of presentation with septic shock.

Some embodiments of the methods include administering a treatment including one or more high risk therapy to a patient that is classified as high risk, or administering a treatment excluding a high risk therapy to a patient that is not high risk, or to provide a method of treating a pediatric patient with septic shock. In some embodiments, the one or more high risk therapy can include at least one selected from: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies. In some embodiments, the biological and/or immune enhancing therapy can include administration of GM-CSF, Interleukin-1 receptor antagonist, Interleukin-7, RUNX1 modulation, and/or anti-PD-1.

In some embodiments, the patient can be enrolled in a clinical trial. In some embodiments, the patient can be classified as high risk. In some embodiments, the method can include predictive enrichment through enrollment of the high risk patient in the clinical trial.

Some embodiments of the methods can include administering a treatment including one or more high risk therapy to the patient in the clinical trial. Some embodiments of the methods further include improving an outcome in a pediatric patient with septic shock.

Some embodiments of the methods further include obtaining a second sample from the treated patient at a second time point; analyzing the second sample to determine gene expression levels of two or more biomarkers selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8; determining whether the gene expression levels of each of the at least two biomarkers are differentially expressed normalized gene expression levels; classifying the patient as high risk of persistent MODS trajectory and/or mortality, or other than high risk of persistent MODS trajectory and/or mortality, based on the determination of whether the expression levels of each of the biomarkers are differentially expressed normalized gene expression levels; and maintaining the treatment being administered if the patient's high risk classification has not changed, or changing the treatment being administered if the patient's high risk classification has changed.

In some embodiments, the second time point can be at least 18 hours after the first time point. In some embodiments, the second time point is in the range of 24 to 96 hours, or longer, after the first time point. In some embodiments, the second time point is about 1 day, 2 days, 3 days, or longer, after the first time point. In some embodiments, the second time point is about 2 days after the first time point. In some embodiments, the first time point is at day 1, wherein day 1 is within 24 hours of a septic shock diagnosis, and the second time point is at day 3. In some embodiments, a patient classified as high risk after the second time point can be administered one or more high risk therapy. In some embodiments, the one or more high risk therapy can include at least one selected from: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies. In some embodiments, the one or more high risk therapy can include a biological and/or immune enhancing therapy.

In some embodiments, a patient not classified as high risk after the second time point can be administered a treatment excluding a high risk therapy. In some embodiments, the patient classified as high risk and administered one or more high risk therapy after the first time point is not classified as high risk after the second time point.

Some embodiments of the methods further include receiving a sample dataset, wherein the sample dataset includes mRNA from a subject having MODS or from a MODS cohort, and analyzing the sample dataset by a machine learning model to identify two or more genes associated with a persistent MODS trajectory and/or mortality.

In some embodiments, the methods can be used as part of a companion diagnostic.

Some embodiments of the disclosure encompass diagnostic kits, tests, or arrays including a reporter hybridization probe, and a capture hybridization probe specific for each of two or more mRNA, DNA, or protein biomarkers selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8.

In some embodiments, the biomarkers include three or more selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8. In some embodiments, the biomarkers include RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8. In some embodiments, the biomarkers include RETN, ADAMTS3, LDHA, LCN2, IL1R2, DDIT4, CEACAM8, MERTK, MPO, and ARL4A.

In some embodiments, the diagnostic kits, tests, or arrays further include a collection cartridge for immobilization of the hybridization probes. In some embodiments, the reporter and the capture hybridization probes include signal and barcode elements, respectively.

Some embodiments of the disclosure encompass compositions including a reporter hybridization probe, and a capture hybridization probe specific for each of two or more biomarkers selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8. In some embodiments, the biomarkers include three or more selected from: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8. In some embodiments, the biomarkers include RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8. In some embodiments, the biomarkers include RETN, ADAMTS3, LDHA, LCN2, IL1R2, DDIT4, CEACAM8, MERTK, MPO, and ARL4A.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts a block diagram illustrating a computer system 100 upon which embodiments of the present teachings may be implemented.

FIG. 2 depicts results from preprocessing of expression measurements belonging to an example derivation dataset. FIG. 2A and FIG. 2B show the effect of normalization on average gene expression values. The x-axis represents the samples, and the y-axis represents the gene expression values. Based on the figures, the average expression values of the samples were more stable and consistent after normalization and suitable for analysis. FIG. 2C shows the association of surrogate variables with the actual batch variable. Since the samples were processed at different time points spread over six years, the resulting variation (batch effect) was removed from the data using, in this example, the ComBat function in the “sva” package in R. The figure shows the association between one of the inferred batch effects through SVA and the actual batch variable (year). The full model (without any batch variable) and the batch variable were passed as separate arguments to the ComBat function. The output consists of a corrected expression set with the batch effects removed completely.

FIG. 3 depicts a workflow summary of example supervised machine learning approaches deployed in the study as described herein, including feature selection and model fitting phases to predict risk of persistent MODS in children with sepsis. Example variable selection methods included Least Absolute Shrinkage and Selection Operator (LASSO), Minimum redundancy and maximum relevance (MRMR), and Random Forest based variable importance technique.

FIG. 4 depicts a summary of model performance determined based on Matthews Correlation Coefficient across four external validation cohorts.

FIG. 5 depicts results from 568 genes found to be differentially expressed among patients with persistent MODS trajectory relative to those without, with 369 genes being upregulated, and 199 genes downregulated. FIG. 5A) Heat map. FIG. 5B) Volcano plot of differentially expressed genes (DEGs) among patients with persistent MODS trajectory vs. those with resolving or no MODS in the training dataset.

FIG. 6 depicts biological pathways enriched among patients with a persistent MODS trajectory relative to those with resolving or no MODS. The size of the circles indicates the number of genes identified per pathway. The gradient shows the adjusted p value for association between pathway and outcome of interest, with red indicating p=<0.005.

FIG. 7 depicts results of CIBERSORT analyses that show differences in proportions of major immune cell subsets among patients with persistent MODS trajectory vs. those with resolving or no MODS. Double asterisk denotes cell types with statistically significant differences between groups of interest.

FIG. 8 depicts the area under the receiver operating characteristic curve (AUROC) for the training cohort and the classifier model. FIG. 8A) AUROC for the risk prediction model to estimate risk of persistent MODS in training cohort. FIG. 8B) AUROC for a fixed 20 gene and Extra Trees classifier model estimating risk of MODS across validation and test sets.

FIG. 9 depicts the area under the receiver operating characteristic curve (AUROC) for day 3 and 7 cardiovascular, respiratory, and renal dysfunction in the derivation cohort.

FIG. 10 depicts protein-protein interaction networks for day 3 cardiovascular, respiratory, and renal dysfunction in the derivation cohort.

FIG. 11. FIG. 11A) Hierarchical clustering of newly derived patient subclasses, FIG. 11B) principal component analyses of subclasses, and FIG. 11C) log rank survival curves of patient subclasses.

FIG. 12A-E depicts the differential expression of 50 genes used to derive patient subclasses between M1 and M2 MODS endotypes. **Indicates statistically significant differences (Wilcox Test, p<0.05).

FIG. 13 depicts a comparison of established pediatric septic shock endotypes (endotypes A and B) and the newly derived patient subclasses (M1, M2, M3, and M4).

### DETAILED DESCRIPTION

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US13/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US13/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US14/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTYPING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTYPING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; U.S. Provisional Application No. 62/764,831, ENDOTYPE TRANSITIONS DURING THE ACUTE PHASE OF PEDIATRIC SEPTIC SHOCK REFLECT CHANGING RISK AND TREATMENT RESPONSE, filed on Aug. 15, 2018; U.S. Provisional Application No. 63/149,744, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2021; International Patent Application No. PCT/US2022/016642, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2022; U.S. Provisional Application No. 63/347,504, PREDICTING PERSISTENT MULTIPLE ORGAN DYSFUNCTION IN THE PEDIATRIC POPULATION AFTER CARDIOPULMONARY BYPASS USING SEPSIS PROGNOSTIC BIOMARKERS, filed on May 31, 2022; and U.S. Provisional Application No. 63/347,944, PEDIATRIC SEPSIS MULTIPLE ORGAN DYSFUNCTION SYNDROME RISK PREDICTION MODEL, filed on Jun. 1, 2022.

Unless otherwise noted, terms are to be understood according to conventional usage by those of ordinary skill in the relevant art.

As used herein, the term “sample” can encompass a sample obtained from a subject or patient. The sample can be of any biological tissue or fluid. Such samples can include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” can include any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to septic shock can refer to a method or process of determining the severity or degree of septic shock or stratifying septic shock based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to organ dysfunction and/or death after septic shock. In some embodiments, “outcome” can refer to two or more organ dysfunctions or death by day 7 of septic shock. In some embodiments, “outcome” can refer to day 7 cardiovascular, respiratory, renal, hepatic, hematologic, and neurologic dysfunction. In some embodiments, “outcome” can refer to 28-day survival/mortality. The importance of survival/mortality in the context of pediatric septic shock is readily evident. The common choice of 28 days was based on the fact that 28-day mortality is a standard primary endpoint for interventional clinical trials involving critically ill patients. In some embodiments, an increased risk for a poor outcome indicates that a therapy has had a poor efficacy, and a reduced risk for a poor outcome indicates that a therapy has had a good efficacy. In some embodiments, “outcome” can refer to resolution of organ failure after 14 days or 28 days or limb loss. Although mortality/survival is obviously an important outcome, survivors have clinically relevant short- and long-term morbidities that impact quality of life, which are not captured by the dichotomy of “alive” or “dead.”

In the absence of a formal, validated quality of life measurement tool for survivors of pediatric septic shock, resolution of organ failure can be used as a secondary outcome measure. For example, the presence or absence of new organ failure over one or more timeframes can be tracked. Patients having organ failure beyond 28 days are likely to survive with significant morbidities having negative consequences for quality of life. Organ failure is generally defined based on published and well-accepted criteria for the pediatric population [18]. Specifically, cardiovascular, respiratory, renal, hepatic, hematologic, and neurologic failure can be tracked. In addition, limb loss can be tracked as a secondary outcome. Although limb loss is not a true “organ failure,” it is an important consequence of pediatric septic shock with obvious impact on quality of life.

As used herein, “outcome” can also refer to complicated course. Complicated course as defined herein relates to persistence of two or more organ failures at day seven of septic shock or 28-day mortality.

As used herein, the terms “predicting outcome” and “outcome risk stratification” with reference to septic shock can refer to a method or process of predicting and/or prognosticating a patient's risk of a certain outcome. In some embodiments, predicting an outcome can relate to monitoring the therapeutic efficacy of a treatment being administered to a patient. In some embodiments, predicting an outcome can relate to determining a relative risk of an adverse outcome (e.g. complicated course) and/or mortality. In some embodiments, the predicted outcome can be associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present disclosure, adverse outcome risk can be determined via the biomarker-based MODS and/or mortality risk stratification as described herein. In some embodiments, predicting an outcome relates to determining a relative risk of MODS and/or mortality. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” can refer to one in which the test agent has “more than minimal risk” (as defined by the terminology used by institutional review boards, or IRBs). In some embodiments, a high risk clinical trial is a drug trial.

As used herein, the term “low risk clinical trial” can refer to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” can refer to any member of the animal kingdom. In some embodiments, a subject is a human patient. In some embodiments, a subject is a pediatric patient. In some embodiments, a pediatric patient is a patient under 18 years of age, while an adult patient is 18 or older. Unless stated otherwise, the terms “patient” or “child” (or “patients” or “children”) refer to a pediatric patient (i.e., under 18 years old).

As used herein, the terms “treatment,” “treating,” “treat,” and the like, can refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” can refer to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” can refer, for example, to a determined level of biomarker expression. The term “pattern of expression levels” can refer to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” can refer to any pattern of expression levels that can be used for the comparison to another pattern of expression levels. In some embodiments of the disclosure, a reference pattern of expression levels is, for example, an average pattern of expression levels observed in a group of healthy or diseased individuals, serving as a reference group.

As used herein, the term “decision tree” can refer to a standard machine learning technique for multivariate data analysis and classification. Decision trees can be used to derive easily interpretable and intuitive rules for decision support systems.

### Overview

Multiple organ dysfunction syndrome (MODS) is a heterogeneous syndrome whose biology is complex, dynamic, and incompletely understood. Elucidating the biological underpinnings of MODS can facilitate the discovery and deployment of targeted therapies, and high throughput ‘omic’ approaches that shed light on immune dysregulation among patients with MODS, regardless of inciting cause, can ultimately lead to strategies that improve patient outcomes.

Over the previous two decades, numerous studies have evaluated gene-expression profiles among critically ill patients to discover a sepsis signature as well as to distinguish sepsis non-survivors from survivors [8-11]. Several have led to identification of genes or related protein biomarkers that have been useful to predict those at highest risk of sepsis mortality [12,13]. Further, unsupervised hierarchical clustering of these genes have been used to determine subclasses or ‘endotypes’ of sepsis [8-10,14], of which those with a dysregulated adaptive immune response have demonstrated differential response to receipt of corticosteroids [15,16]. If prospectively validated, gene-expression based predictive enrichment strategies can be used to personalize therapies for patients with sepsis.

Few transcriptomic studies have explicitly focused on MODS as the primary outcome [17-20]. Given the dynamic nature of sepsis and substantial morbidity associated with persistence of MODS, focusing on this subset of patients can lead to advancements in their care.

To address the significant underlying heterogeneity associated with MODS, the disclosure demonstrates that predictive enrichment explicitly focused on distinguishing organ dysfunction trajectories can unravel the underlying biology and advance patient endotyping and therefore treatment. Machine learning (ML), which has been previously used by the present inventors to determine gene-expression signatures correlated with the static endpoint of complicated course [18], has been used as described herein to facilitate identification of gene-expression signatures and endotypes correlated with multiple organ dysfunction trajectories among children with sepsis.

As described herein, gene expression signatures associated with MODS trajectories can facilitate prediction of at-risk patients, inform their underlying biology, as well as identification of molecular targets and predictive enrichment. Secondary analyses of publicly available datasets were conducted, using supervised ML to identify a parsimonious set of genes associated with a persistent MODS trajectory in a training set of pediatric septic shock. Model parameters were determined and risk-prediction capabilities were independently tested across test datasets, and in relation to established gene-sets predictive of sepsis mortality.

In the study described herein, publicly available datasets have been leveraged to identify the gene-signatures associated with a persistent MODS trajectory among critically ill patients and unravel biological mechanisms at play. Supervised machine learning (ML) approaches were implemented to identify a parsimonious set of genes predictive of the outcome of interest; these approaches were trained and validated in a model to reliably identify those at high risk of MODS, and reproducibility of this approach was demonstrated across test datasets irrespective of the cause of organ dysfunctions.

Specifically, patients with a persistent MODS trajectory were found to have 568 differentially expressed genes, and were characterized by a dysregulated innate immune response. Supervised ML identified 111 features consistently associated with outcome of interest with an AUROC of 0.87 (95% CI: 0.85-0.88) in the training cohort. Model performance using the top 20 genes and an ExtraTree classification model yielded AUROCs ranging 0.77-0.96 among validation cohorts. Genes correlated with day 3 and 7 cardiovascular, respiratory, and renal dysfunctions were identified. The refined model, limited to 20 genes (RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8), achieved AUROCs ranging from 0.74-0.79 in the validation and test cohorts to predict those with MODS across pediatric and adult datasets, agnostic to the cause of organ dysfunctions.

Model performances were tested across 4 validation cohorts, among children and adults with differing inciting cause for organ dysfunctions, to identify a stable set of genes and fixed classification model to reliably estimate the risk of MODS. Clinical propensity scores, where available, were used to enhance model performance. Organ-specific dysfunction signatures were identified by eliminating redundancies between the shared MODS signature and those of individual organ dysfunctions. Finally, novel patient subclasses were identified through unsupervised hierarchical clustering of genes correlated with persistent MODS and compared with previously established pediatric septic shock endotypes.

The top 50 genes—namely GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, and RUNX1—were used to discover four novel subclasses, of which patients belonging to M1 and M2 had the worst clinical outcomes. Reactome pathway analyses revealed the role of transcription factor RUNX1 in distinguishing subclasses. Interaction with receipt of adjuvant steroids indicated that newly derived M1 and M2 endotypes were biologically distinct relative to previously established endotypes.

This study also determined whether the limited set of genes identified as described herein improved upon previously published gene sets that have been demonstrated to predict sepsis mortality, in identifying patients with MODS. Indeed, the model identified and described herein demonstrated greater reproducibility in identifying those with MODS, relative to published gene-sets predictive of sepsis mortality.

Thus, the disclosure provides evidence for a unique gene-expression signature associated with persistent MODS trajectory. This gene-expression signature can be extended to clinical practice, as a determination of patient gene expression levels from the group of the top 50 genes (GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and/or MMP8) can be used to classify a patient with septic shock as high risk of persistent MODS trajectory and/or mortality or other than high risk of persistent MODS trajectory and/or mortality. The determination of patient gene expression levels can include some or all of the 20 genes of the further refined model, i.e. RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8, to provide a classification of a patient with septic shock as high risk of persistent MODS trajectory and/or mortality or other than high risk of persistent MODS trajectory and/or mortality.

When the gene expression levels of the biomarkers are differentially expressed normalized gene expression levels, the patient can be classified as high risk of persistent MODS trajectory and/or mortality, and an appropriate treatment can be determined and administered. For example, a patient that is classified as high risk can be administered a treatment comprising one or more high risk therapy, such as, for example, biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies, etc. In contrast, a patient that is not high risk can be administered a treatment excluding a high risk therapy. This allows for more effective and personalized treatment, with improved outcomes.

This can be particularly useful when selecting a patient for a high risk treatment and/or for a high risk clinical trial, as only a patient classified as high risk of persistent MODS trajectory and/or mortality should be selected for a high risk treatment and/or enrolled in a high risk clinical trial. A patient who is not classified as high risk of persistent MODS trajectory and/or mortality should not be administered a high risk treatment and/or enrolled in a high risk clinical trial.

This study demonstrated the utility of supervised ML analyses of transcriptomic datasets to reliably identify patients at risk of MODS. Combined with validation in enriched cohorts with a high burden of organ dysfunctions, this gene-expression classifier can facilitate the early identification of high-risk critically ill patients who may benefit from targeted therapies, including those that modulate the innate immune response.

The studies described herein provide evidence that machine learning can be used to optimize feature selection to reliably identify those at risk of multiple and individual organ dysfunctions and delineate patient subclasses with vastly different clinical outcomes. These data demonstrate the existence of complex sub-endotypes among children with septic shock wherein overlapping biological pathways are linked to differential response to therapies and clinical trajectories. Future studies in cohorts enriched for patients with MODS can inform the underlying biology and facilitate discovery and development of novel or repurposed disease modifying therapies for subsets of critically ill children with sepsis.

Accordingly, the biomarkers identified and described herein can be used as biomarkers for “predictive” enrichment, i.e., to identify biologically relevant subpopulations of disease with the ultimate intent of discovering targeted therapies, and have prognostic utility as well. In comparison, many previous studies help with prognostic enrichment. The biomarkers described herein can thus be used for predicting and/or estimating risk of outcomes, such as mortality or multiple organ dysfunctions. Further, predicting and/or estimating risk of outcomes, such as mortality or multiple organ dysfunctions, can be used to identify and administer an appropriate treatment for an individual patient, depending on the prediction and/or estimated risk.

### Additional Patient Information

The demographic data, clinical characteristics, and/or results from other tests or indicia of septic shock specific to a pediatric patient with MODS, persistent MODS trajectory, and/or mortality risk can affect the patient's outcome risk. Accordingly, such demographic data, clinical characteristics, and/or results from other tests or indicia of septic shock can be incorporated into the methods described herein which allow for stratification of individual pediatric patients in order to determine the patient's outcome risk. Such demographic data, clinical characteristics, and/or results from other tests or indicia of septic shock can also be used in combination with the methods described herein which allow for stratification of individual pediatric patients in order to determine the patient's outcome risk.

Such pediatric patient demographic data can include, for example, the patient's age, race, gender, and the like. In some embodiments, the biomarker-based MODS and/or mortality risk stratification described herein can incorporate or be used in combination with the patient's age, race, and/or gender to determine an outcome risk.

Such patient clinical characteristics and/or results from other tests or indicia of septic shock can include, for example, the patient's co-morbidities and/or septic shock causative organism, and the like.

Patient co-morbidities can include, for example, acute lymphocytic leukemia, acute myeloid leukemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NYHA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient.

Septic shock causative organisms can include, for example, Acinetobacter baumannii, Adenovirus, Bacteroides species, Candida species, Capnotyophaga jenuni, Cytomegalovirus, Enterobacter cloacae, Enterococcus faecalis, Escherichia coli, Herpes simplex virus, Human metapneumovirus, Influenza A, Klebsiella pneumonia, Micrococcus species, mixed bacterial infection, Moraxella catarrhalis, Neisseria meningitides, Parainfluenza, Pseudomonas species, Serratia marcescens, Staphylococcus aureus, Streptococcus agalactiae, Streptococcus milleri, Streptococcus pneumonia, Streptococcus pyogenes, unspecified gram negative rods, unspecified gram positive cocci, and the like.

In some embodiments, the biomarker-based MODS, persistent MODS trajectory, and/or mortality risk stratification as described herein can incorporate the patient's co-morbidities to determine an outcome risk and/or mortality probability. In some embodiments, the biomarker-based MODS, persistent MODS trajectory, and/or mortality risk stratification as described herein can incorporate the patient's septic shock causative organism to determine an outcome risk and/or mortality probability.

In some embodiments, the biomarker-based MODS, persistent MODS trajectory, and/or mortality risk stratification as described herein can be used in combination with the patient's co-morbidities to determine an outcome risk and/or mortality probability. In some embodiments, the biomarker-based MODS, persistent MODS trajectory, and/or mortality risk stratification as described herein can be used in combination with the patient's septic shock causative organism to determine an outcome risk and/or mortality probability.

### PERSEVERE and Other Population-Based Risk Scores

The PERSEVERE model for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE model is based on Interleukin-8 (IL-8), Heat shock protein 70 kDA (HSP70), C-C Chemokine ligand 3 (CCL3), C-C Chemokine ligand 4 (CCL4), Granzyme B (GZMB), Interleukin-1 α (IL-1a), and Matrix metallopeptidase 8 (MMP8). PERSEVERE additionally takes patient age into account.

The PERSEVERE decision tree has 8 terminal nodes. Of these, 3 terminal nodes of the PERSEVERE decision tree are determined to be low risk/low mortality probability (terminal nodes 2, 4, and 7), while 5 terminal nodes of the PERSEVERE decision tree are determined to be intermediate to high risk/high mortality probability (terminal nodes 1, 3, 5, 6, and 8). In some embodiments, a low risk/low mortality probability terminal node has a mortality probability between 0.000 and 0.025, while an intermediate to high risk/high mortality probability terminal nodes has a mortality probability greater than 0.025.

In some embodiments of the present disclosure, the PERSEVERE mortality probability stratification can be used in combination with biomarker-based MODS and/or mortality risk stratification as described herein. In some embodiments, the biomarker-based MODS and/or mortality risk stratification, as described herein, can be used in combination with a patient endotyping strategy and/or Z score determination. In some embodiments, the combination of a biomarker-based MODS and/or mortality risk stratification, with an endotyping strategy and/or Z score determination, can be used to determine an appropriate treatment regimen for a patient. For example, such combinations can be used to identify which patients are more likely to benefit from corticosteroids.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for and are not used for stratification of individual patients. The methods described herein which allow for stratification of individual patients can be used alone or in combination with one or more existing population-based risk scores.

In some embodiments, the biomarker-based MODS and/or mortality risk stratification described herein can be used with one or more additional population-based risk scores. In some embodiments, the biomarker-based MODS and/or mortality risk stratification described herein can be used in combination with PRISM. In some embodiments, the biomarker-based MODS and/or mortality risk stratification described herein can be used in combination with PIM. In some embodiments, the biomarker-based MODS and/or mortality risk stratification described herein can be used in combination with PELOD. In some embodiments, the biomarker-based MODS and/or mortality risk stratification described herein can be used in combination with a population-based risk score other than PRISM, PIM, and PELOD.

### High Risk Therapies

High risk, invasive therapeutic and support modalities can be used to treat MODS, mortality risk, and/or persistent MODS trajectory. The methods described herein which allow for the patient's outcome risk to be determined can help inform clinical decisions regarding the application of high risk therapies to specific pediatric patients, based on the patient's outcome risk.

High risk therapies include, for example, adjuvant hemoperfusion, plasma filtration and adsorption therapies, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or high risk therapies. In particular, patients at high risk of MODS and/or mortality risk stratification can be treated with immune enhancing therapies, such as, for example, interleukin-1 receptor antagonist (Anakinra), GMCSF, interleukin-7, anti-PD-1, and the like.

In some embodiments, individualized treatment can be provided to a pediatric patient by selecting a pediatric patient classified as high risk by the methods described herein for one or more high risk therapies. In some embodiments, individualized treatment can be provided to a pediatric patient by excluding a pediatric patient classified as low risk from one or more high risk therapies.

Certain embodiments of the disclosure include using quantification data from a gene-expression analysis and/or from a protein, mRNA, and/or DNA analysis, from a sample of blood, urine, saliva, broncho-alveolar lavage fluid, or the like. Embodiments of the disclosure include not only methods of conducting and interpreting such tests but also include reagents, compositions, kits, tests, arrays, apparatuses, processing devices, assays, and the like, for conducting the tests. The compositions and kits of the present disclosure can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a pediatric patient with septic shock.

The correlations disclosed herein, between pediatric patient MODS, persistent MODS trajectory, and/or mortality risk biomarker levels and/or mRNA levels and/or gene expression levels, and/or protein expression levels, provide a basis for conducting a diagnosis of MODS, persistent MODS trajectory, and/or mortality risk, or for conducting a stratification of patients with MODS, persistent MODS trajectory, and/or mortality risk, or for enhancing the reliability of a diagnosis of MODS, persistent MODS trajectory, and/or mortality risk by combining the results of a quantification of a MODS, persistent MODS trajectory, and/or mortality risk biomarker with results from other tests or indicia of MODS, persistent MODS trajectory, and/or mortality risk, or for determining an appropriate treatment regimen for a pediatric patient with MODS, persistent MODS trajectory, and/or mortality risk. For example, the results of a quantification of one biomarker could be combined with the results of a quantification of one or more additional biomarker, protein, cytokine, mRNA, or the like. Thus, even in situations in which a given biomarker correlates only moderately or weakly with septic shock, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the disclosure are expressly contemplated to be used both alone and in combination with other tests and indicia, whether quantitative or qualitative in nature.

### Dosage and Administration Routes

Other embodiments of the disclosure can include methods of administering or treating an animal, which can involve administering an amount of at least one treatment that is effective to treat the disease, condition, or disorder that the organism has, or is suspected of having, or is susceptible to, or to bring about a desired physiological effect. In some embodiments, the composition or pharmaceutical composition comprises at least one treatment, which can be administered to an animal (e.g., mammals, primates, monkeys, or humans) in an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some subjects (e.g., mammals, mice, rabbits, feline, porcine, or canine) can be administered a dosage of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present disclosure, and using, in part, the guidance provided herein, will be able to adjust and test any number of concentrations in order to find one that achieves the desired result in a given circumstance. In some embodiments, a dose or a therapeutically effective dose of a compound disclosed herein will be that which is sufficient to achieve a plasma concentration of the compound or its active metabolite(s) within a range set forth herein, e.g., about 1-10 nM, 10-100 nM, 0.1-1 μM, 1-10 μM, 10-100 μM, 100-200 μM, 200-500 μM, or even 500-1000 μM, preferably about 1-10 nM, 10-100 nM, or 0.1-1 μM.

In other embodiments, a treatment can be administered in combination with one or more other therapeutic agents for a given disease, condition, or disorder.

The compounds and pharmaceutical compositions are preferably prepared and administered in dose units. Solid dose units are tablets, capsules and suppositories. For treatment of a subject, depending on activity of the compound, manner of administration, nature and severity of the disease or disorder, age and body weight of the subject, different daily doses can be used.

Under certain circumstances, however, higher or lower daily doses can be appropriate. The administration of the daily dose can be carried out both by single administration in the form of an individual dose unit or else several smaller dose units and also by multiple administrations of subdivided doses at specific intervals.

A treatment can be administered locally or systemically in a therapeutically effective dose. Amounts effective for this use will, of course, depend on the severity of the disease or disorder and the weight and general state of the subject. Typically, dosages used in vitro can provide useful guidance in the amounts useful for in situ administration of the pharmaceutical composition, and animal models can be used to determine effective dosages for treatment of particular disorders.

Various considerations are described, e. g., in Langer, 1990, Science, 249: 1527; Goodman and Gilman's (eds.), 1990, Id., each of which is herein incorporated by reference and for all purposes. Dosages for parenteral administration of active pharmaceutical agents can be converted into corresponding dosages for oral administration by multiplying parenteral dosages by appropriate conversion factors. As to general applications, the parenteral dosage in mg/mL times 1.8=the corresponding oral dosage in milligrams (“mg”). As to oncology applications, the parenteral dosage in mg/mL times 1.6=the corresponding oral dosage in mg. An average adult weighs about 70 kg. See e.g., Miller-Keane, 1992, Encyclopedia & Dictionary of Medicine, Nursing & Allied Health, 5th Ed., (W. B. Saunders Co.), pp. 1708 and 1651.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In some embodiments, the administration can include a unit dose of one or more treatments in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate. [00275] The quantity of active component in a unit dose preparation can be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

A treatment can be administered to subjects by any number of suitable administration routes or formulations. The treatment can also be used to treat subjects for a variety of diseases. Subjects include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject”, unless stated otherwise, encompasses both human and non-human subjects.

The route of administration of the compounds of the treatments described herein can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease (e.g., type of cancer), and the severity of the disease (e.g., stage or severity of cancer). Of course, combinations of administration routes can be administered, as desired.

Some embodiments of the disclosure include a method for providing a subject with a treatment which comprises one or more administrations of one or more compositions; the compositions may be the same or different if there is more than one administration.

### Toxicity

The ratio between toxicity and therapeutic effect for a particular treatment is its therapeutic index and can be expressed as the ratio between LD50 (the amount of compound lethal in 50% of the population) and ED50 (the amount of compound effective in 50% of the population). Compounds that exhibit high therapeutic indices are preferred. Therapeutic index data obtained from in vitro assays, cell culture assays and/or animal studies can be used in formulating a range of dosages for use in humans. The dosage of such compounds preferably lies within a range of plasma concentrations that include the ED50 with little or no toxicity. The dosage can vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual practitioner in view of the patient's condition and the particular method in which the compound is used. For in vitro formulations, the exact formulation and dosage can be chosen by the individual practitioner in view of the patient's condition and the particular method in which the compound is used.

### Computer Implemented System

In various embodiments, the systems and methods for classifying a patient with septic shock as high risk of persistent MODS trajectory and/or mortality or other than high risk of persistent MODS trajectory and/or mortality can be implemented via computer software or hardware.

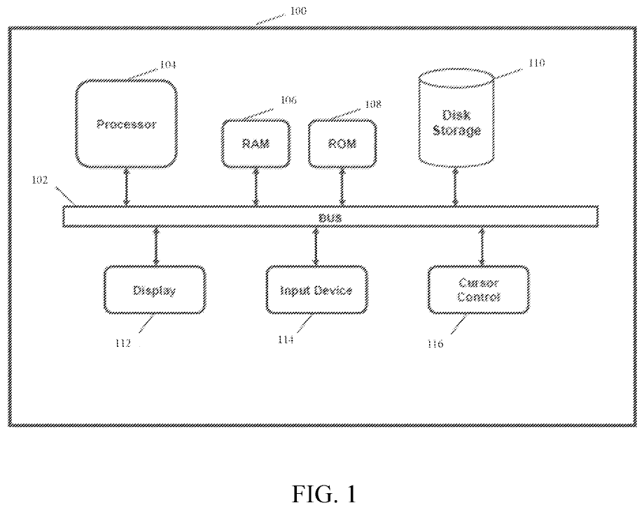

FIG. 1 is a block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communication of information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 100 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 100, whereby processor 104 would execute the analyses and determinations provided by these engines, subject to instructions provided by any one of, or a combination of, memory components 106/108/110 and user input provided via input device 114.

In various embodiments, the methods of the present teachings can involve deep learning and/or machine learning and/or one or more neural network, such as a deep neural network, and the like. It should be understood that while deep learning and such processes may be discussed in conjunction with various embodiments herein, the various embodiments herein are not limited to being associated only with deep learning tools. As such, machine learning and/or artificial intelligence tools generally may be applicable as well. Moreover, the terms deep learning, machine learning, and artificial intelligence may even be used interchangeably in generally describing the various embodiments of systems, software and methods herein.

A deep neural network (DNN) generally, such as a convolutional neural network (CNN), generally accomplishes an advanced form of image processing and classification/detection by first looking for low level features such as, for example, edges and curves, and then advancing to more abstract (e.g., unique to the type of images being classified) concepts through a series of convolutional layers. A DNN/CNN can do this by passing an image through a series of convolutional, nonlinear, pooling (or downsampling, as will be discussed in more detail below), and fully connected layers, and get an output. Again, the output can be a single class or a probability of classes that best describes the image or detects objects on the image.

Regarding layers in a CNN, for example, the first layer is generally a convolutional layer (Conv). This first layer will process the image's representative array using a series of parameters. Rather than processing the image as a whole, a CNN will analyze a collection of image sub-sets using a filter (or neuron or kernel). The sub-sets will include a focal point in the array as well surrounding points. For example, a filter can examine a series of 5×5 areas (or regions) in a 32×32 image. These regions can be referred to as receptive fields. Since the filter must possess the same depth of the input, an image with dimensions of 32×32×3 would have a filter of the same depth (e.g., 5×5×3). The actual step of convolving, using the exemplary dimensions above, would involve sliding the filter along the input image, multiplying filter values with the original pixel values of the image to compute element wise multiplications, and summing these values to arrive at a single number for that examined portion of the image.

After completion of this convolving step, using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents (e.g., colors, edges, curves, etc.) that, together, represent the feature identifiers required for the final image output. These filters, when used in combination, allow the CNN to process an image input to detect those features present at each pixel. Therefore, if a filter serves as a curve detector, the convolving of the filter along the image input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Cony, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Conv. Therefore, the choice and number of filters (or channels) to meet the needs of the CNN method are specifically chosen to produce as accurate an output as possible while considering the time and power available.

To enable further a CNN to detect more complex features, additional Conv layers can be added to analyze what outputs from the previous Conv layer (i.e., activation maps). For example, if a first Conv layers looks for a basic feature such as a curve or an edge, a second Conv layer can look for a more complex feature such as shapes, which can be a combination of individual features detected in an earlier Conv layer. By providing a series of Conv layers, the CNN can detect increasingly higher-level features to arrive eventually at the specific desired object detection. Moreover, as the Conv layers stack on top of each other, analyzing the previous activation map output, each Conv layer in the stack is naturally going to analyze a larger and larger receptive field by virtue of the scaling down that occurs at each Conv level, thereby allowing the CNN to respond to a growing region of pixel space in detecting the object of interest.

A CNN architecture generally consists of a group of processing blocks, including at least one processing block for convoluting an input volume (image) and at least one for deconvolution block (or transpose convolution). Additionally, the processing blocks can include at least one pooling block and unpooling block. Pooling blocks can be used to scale down an image in resolution to produce an output available for Conv. This can provide computational efficiency (efficient time and power), which can in turn improve actual performance of the CNN. Those these pooling, or subsampling, blocks keep filters small and computational requirements reasonable, these blocks coarsen the output (can result in lost spatial information within a receptive field), reducing it from the size of the input by a factor equal to the pixel stride of the receptive fields of the output units.

Unpooling blocks can be used to reconstruct a these coarse outputs to produce an output volume with the same dimensions as the input volume. An unpooling block can be considered a reverse operation of a convoluting block to return an activation output to the original input volume dimension.

However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down an image and unpooling blocks can be used to enlarge these scaled down activation maps, convolution and deconvolution blocks can be structured to both convolve/deconvolve and scale down/enlarge without separate pooling and unpooling blocks.

The pooling and unpooling process can be limited depending on the objects of interest being detected in an image input. Since pooling generally scales down an image by looking at sub-image windows without overlap of windows, there is a clear loss in spatial info as the scaling down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv layer in its processing block. The ReLU or ELU layer acts as a gating function to advance only those values corresponding to positive detection of the feature of interest unique to the Conv layer its processing block.

Given a basic architecture, the CNN is then prepared for a training process to hone its accuracy in image classification/detection (of objects of interest). Using training data sets, or sample images used to train the CNN so that it updates its parameters in reaching an optimal, or threshold, accuracy, a process called backpropagation (backprop) occurs. Backpropagation involves a series of repeated steps (training iterations) that, depending on the parameters of the backprop, either will slowly or quickly train the CNN. Backprop steps generally include forward pass, loss function, backward pass, and parameter (weight) update according to a given learning rate. The forward pass involves passing a training image through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the desire for lower learning rates and more specific and carefully determined initial parameters upon start of training. One such complication is that, as weight updates occur at the conclusion of each iteration, the changes to the parameters of the Conv layers amplify the deeper the network goes. For example, if a CNN has a plurality of Conv layers that, as discussed above, allows for higher-level feature analysis, the parameter update to the first Conv layer is multiplied at each subsequent Conv layer. The net effect is that the smallest changes to parameters have large impact depending on the depth of a given CNN. This phenomenon is referred to as internal covariate shift.

It should be noted that even though CNNs are spoken about in detail above, the various embodiments discussed herein could utilize any neural network type or architecture.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

Although specific embodiments and applications of the disclosure have been described in this specification, these embodiments and applications are exemplary only, and many variations are possible. Having described the disclosure in detail, it will be apparent that modifications, variations, and equivalent embodiments are possible without departing from the scope of the disclosure defined in the appended claims. Furthermore, it should be appreciated that all examples in the present disclosure are provided as non-limiting examples.

### EXAMPLES

The following non-limiting examples are provided to further illustrate embodiments of the disclosure disclosed herein. It should be appreciated by those of skill in the art that the techniques disclosed in the examples that follow represent approaches that have been found to function well in the practice of the disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Study Population

De-identified clinical data and publicly available gene-expression datasets were used for the purposes of this study. Phenotyping data of organ dysfunction trajectories based on clinical and laboratory data available between day 1 through 7 of pediatric intensive care unit (PICU) admission were available in the training cohort, as previously detailed [21]. The primary comparison of interest was persistent MODS (death by day 7, persistence of ≥2 organ dysfunctions on day 7, or new MODS between days 1-7), relative to those with resolving MODS (≥2 organ dysfunctions on day 1 or 3 with <2 dysfunctions by day 3 and 7 respectively), and those with no MODS, the latter comprised of septic patients, non-septic patients with systemic inflammatory response syndrome (SIRS), and healthy controls. The choice for this comparison was guided by the fact that patients with persistent organ dysfunctions, despite intensive organ support, are likely to represent a subset of patients who may benefit from innovative targeted therapeutic interventions based on their underlying biological predisposition.

Analyses were conducted with and without inclusion of patients who died within the first 7 days, to test the premise that non-survivors may have a different expression signature relative to survivors with persistent organ dysfunctions. Secondary comparisons focused on those with and without cardiovascular, respiratory, and kidney dysfunction on day 3 and 7 respectively.

### Propensity Matching.

Given the substantial demographic heterogeneity, a propensity score was generated for each patient to account for the confounding influence of age and illness severity, as determined by the PRISM III score [46] for the risk of MODS. Values of 0, 3, and 5 were randomly imputed for PRISM-III scores for controls, in whom these data were not available. R package “MatchIt” was used to perform matching and full propensity match method was used. Each patient received a propensity score, which was used to train the machine learning (ML) models and incorporated into risk prediction models.

### Example 2

### Training Cohort

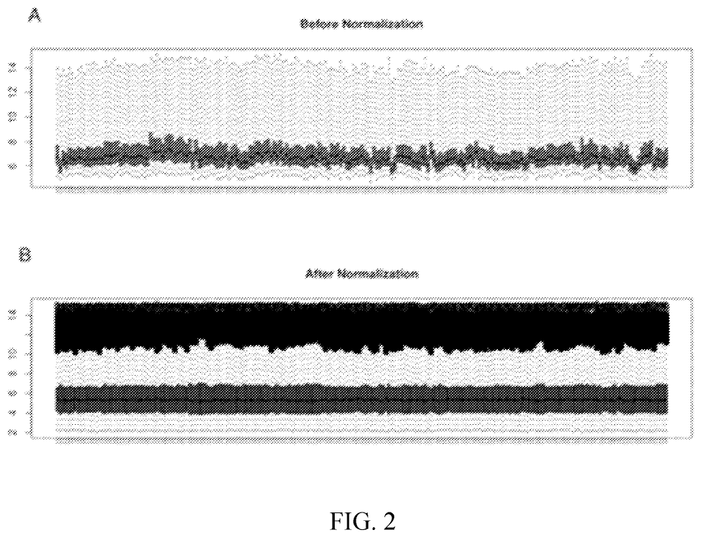

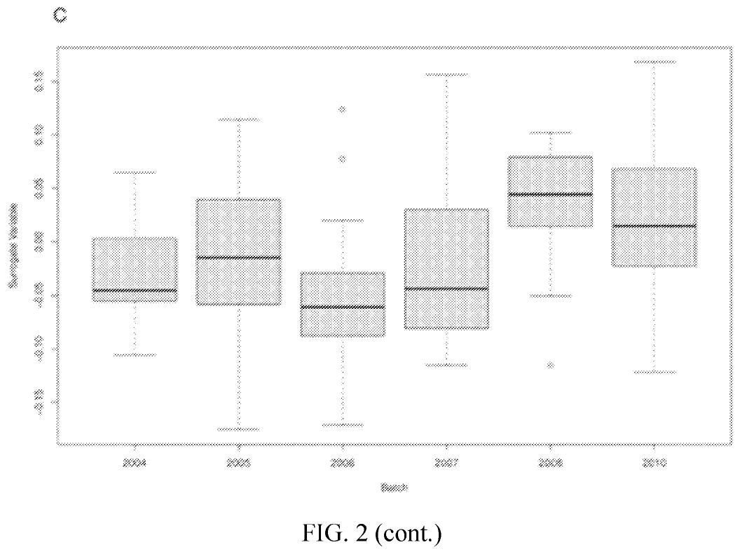

Microarray dataset GSE66099 [22] was downloaded from the NCBI Gene Expression Omnibus (GEO) repository [20] and served as the training dataset. The Affymetrix probes were matched to gene symbols using the Affymetrix Human Genome U133 Plus 2.0 (hgu133plus2.db). Gene expression data were pre-processed including batch correction for year of study, as detailed in Tables 1 and 2 and FIG. 2. This study considered the year of measurement of the gene expression data as the batch variable. Ideally, batch corrections are possible only if the batch variables are not highly correlated with the outcome (MODS in this dataset).

From Table 1, it is clear that a tight correlation between the batch variable (year) and the outcome of interest is absent. Within each batch, there are measurements from multiple different groups, so the batch effect removal process proceeded.

The ‘sva’ package in R was used to identify batch effects in these data. Although there was prior information regarding the batch variable (the year of measurement), the analysis checked if SVA could find new covariates explaining the variation in the data. The ‘sv’ component returned by the sva function contained the two new covariates or the potential batch effects. To check if the new surrogate variables (or SVs) are associated with the observed batch variable, a linear model is fit using the lm( ) in R.

From Table 2, it is observed that the second estimated surrogate variable has a significant correlation with the batch variable. In this case, the coefficient shows that by changing the batch variable, the value of the SV changes by 8.03, and this result is significant (P=9e-05). This shows that the estimated SV is associated with the batch.

Differential expression of genes (DEGs) based on a log 2 fold change≥±0.5, adjusted value for Benjamini Hochberg correction for false discovery rate<0.05, was performed using the limma package in R [23]. Sensitivity analyses were conducted with and without inclusion of patients who died within the first 7 days, to test the premise that non-survivors may have a different signature relative to survivors with persistent organ dysfunctions. The analysis used clusterProfiler [24] for functional gene enrichment, and CIBERSORT [25], a computational tool to deconvolute bulk expression data and estimate abundance of various immune cells subsets.

TABLE 1
Number of gene expression measurements made
for six years for the GSE66099 dataset.

| | 2004 | 2005 | 2006 | 2007 | 2008 | 2010 |
|---|---|---|---|---|---|---|
| Outcome = evolving MODS | 2 | 7 | 5 | 8 | 5 | 17 |
| Outcome = no evolving | 7 | 40 | 14 | 23 | 23 | 50 |
| MODS | | | | | | |

TABLE 2
Results of regressing the surrogate variables returned
by the sva() and the actual batch effects.

| | Coefficients | | | |
|---|---|---|---|---|
| | | | Std. | Significance |
| Formula | Components | Estimate | Error | level |
| Surrogate Variable | Intercept | 2007.45 | 0.146 | <2e−16 |
| 1 ~ Batch variable | Batch | 2.6029 | 2.08 | 0.213 |
| Surrogate Variable | Intercept | 2007.45 | 0.14 | <2e−16 |
| 2 ~ Batch variable | Batch | 8.03 | 2.01 | 9.00E−05 |

### Example 3

### Supervised Machine Learning

The supervised machine learning methods used in the examples described herein are summarized below:

### Feature Selection.

Due to the high dimensionality of the dataset, different feature selection strategies were evaluated to extract a high performing subset of highly discriminative genes to distinguish patients with persistent MODS trajectory, relative to those with resolving or no MODS. Three variable selection techniques were used, including least absolute shrinkage and selection operator (LASSO), minimum redundancy and maximum relevance (MRMR), and random forests (RF)-based variable importance technique. The genes selected by each of the above methods were aggregated into a single input feature set, and the list of DEGs obtained were added to the list. The propensity score for each patient was included in the list of features used to train the classifier.

### Model Fitting.

To counter the class imbalances in the training data, both undersampling and oversampling techniques were incorporated into the training dataset, as described below. Briefly, three binary classifications algorithms were used, including logistic regression and two tree-based classifiers (Random Forest and Extra Trees classifiers).

Among the undersampling techniques, Cluster centroids (CCN), Repeated Edited Nearest Neighbors (REDN), and Random Undersampling (RUS) were implemented. Cluster centroids calculate the centroid of the majority class using the k-means and then find instances nearest to this centroid in the input feature space. As a result, instances that are far away from the centroid are discarded. In the case of Edited Nearest Neighbors (EDN), all samples whose class label differs from that of half of their k-nearest neighbors are removed. Repeated Edited Nearest Neighbors (REDN) applies the EDN technique until no further samples can be discarded from the majority class. Random undersampling (RUS) involves randomly removing samples from the majority class in order to balance the dataset. Among the oversampling techniques, SMOTE (Synthetic Minority Oversampling Technique) works by generating new instances of the minority class from existing data. ADASYN (Adaptive Synthetic) sampling approach is similar to SMOTE and works by generating an appropriate number of synthetic samples belonging to the minority class. Three binary classification algorithms (two tree-based and logistic regression) were used to build the machine learning models. The two tree-based classifiers included Random Forests and Extra Trees classifiers. A random forest classifier works by combining the predictions from hundreds of decision trees built on random bootstrapped samples of the dataset using a random selection of features when splitting the nodes. The extra trees classifier is a meta-estimator that fits an arbitrary number of randomized trees (base models) on different sub-samples of the data based on the user's input. It then combines the predictive power of these base models into one final optimal model. A Logistic Regression (LOGIT) classifier works by calculating a linear combination of the log-transformed expression estimates across samples and generating a linear decision boundary to separate the two classes from one another.

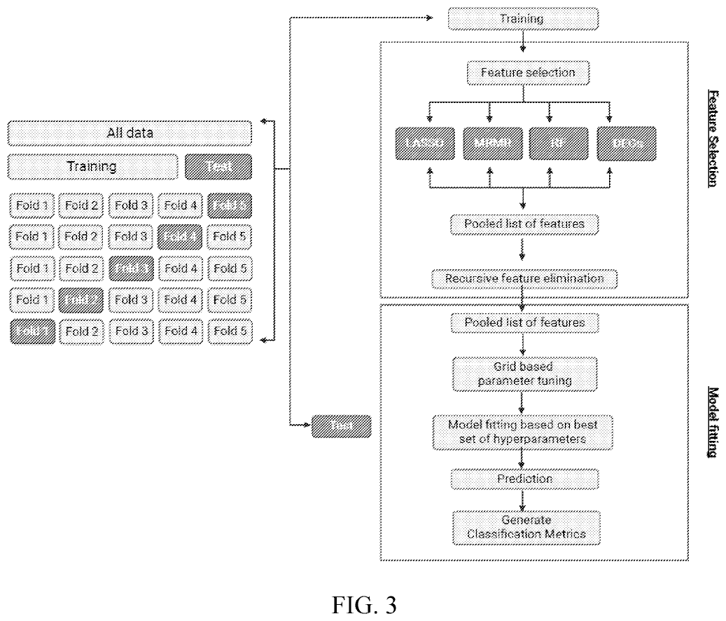

A 5-fold cross-validation process was applied, similar to those previously published by this group [18], that involves randomly partitioning the dataset into five equal subsets in a stratified fashion. Hyper-parameter tuning was done using a cross-validated grid search technique on a subset of the training data over a parameter grid using the area under the curve as the scoring function. The analysis experimented with different classification thresholds from 0 to 1 with step sizes of 0.001, choosing the one that provided the maximum area under the receiver operator characteristic curve (AUROC). To evaluate robustness of the model training and to ensure complete cross-validation, the entire process was repeated seven times, resulting in thirty-five unique train and test splits. The performances obtained during each run were averaged, and the mean scores along with the 95% CI were reported. The overall approach is summarized in FIG. 3.

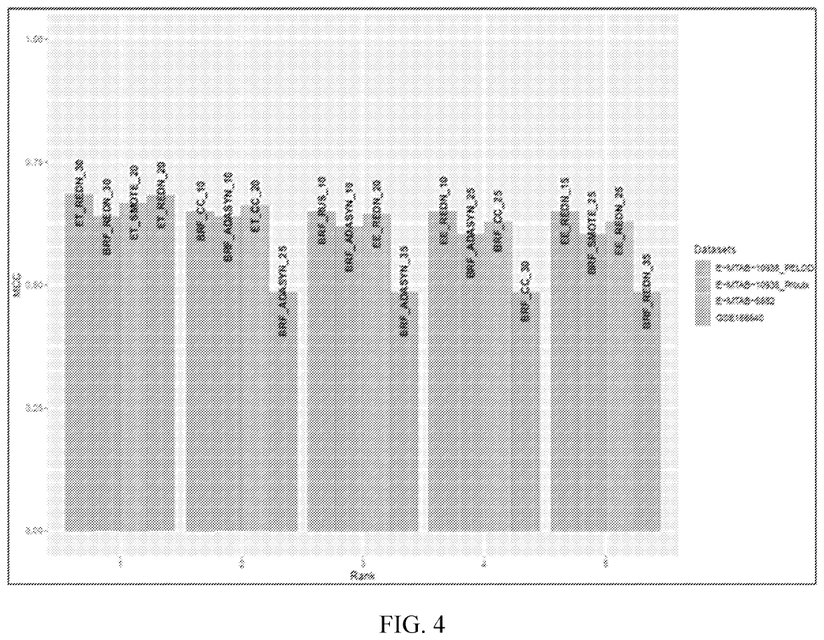

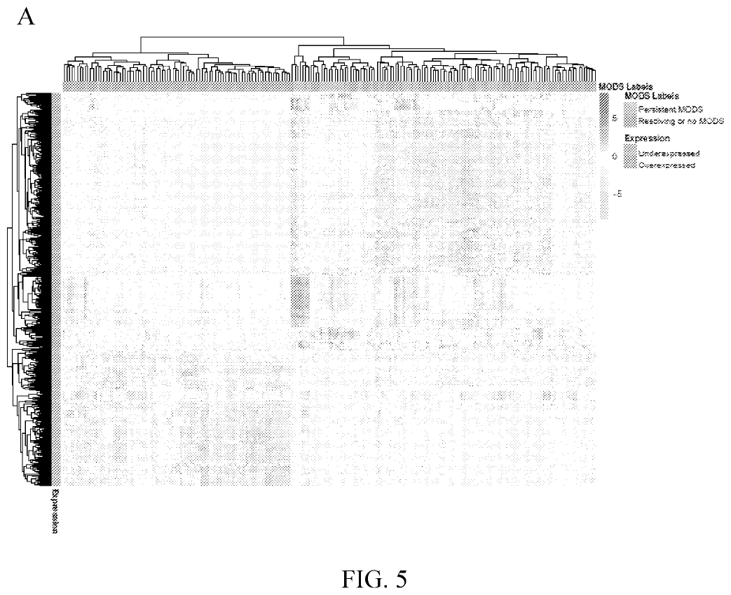

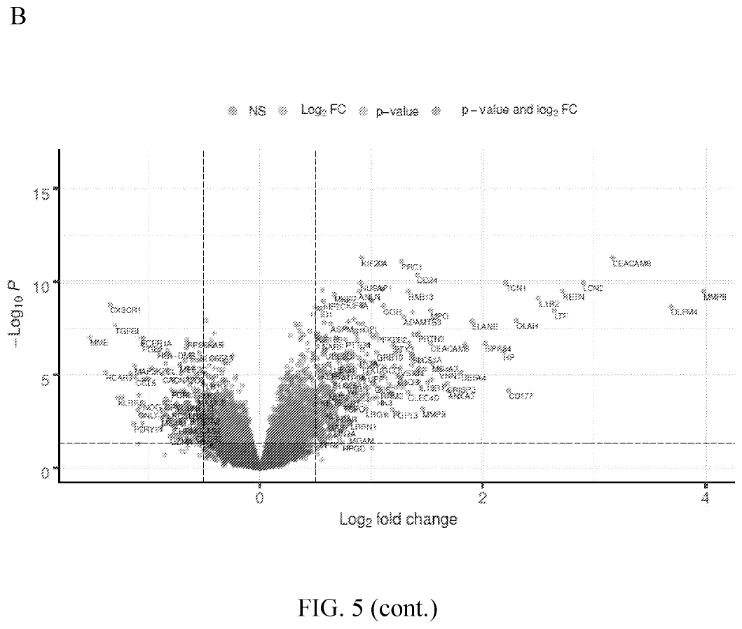

Four out of the five subsets formed the training set, and the remaining subset was used for testing set; the process was repeated until each fold had been evaluated as a test data. In each training phase, we first integrated the features obtained using the three feature selection approaches, balanced the dataset using sampling techniques, and finally applied the recursive feature elimination algorithm to arrive at a list of features that were most relevant in predicting the target variable, as summarized in FIG. 4. The overall learning process that was adopted in this study is similar to the previous analysis aimed at identifying a set of robust biomarkers predictive of a complicated course outcome among pediatric sepsis patients admitted to the ICU and is summarized in FIG. 4. Hyper-parameter tuning was done using a cross-validated grid search technique on a subset of the training data over a parameter grid using the area under the curve as the scoring function. Different classification thresholds were used, from 0 to 1 with step sizes of 0.001, and the one that provided the maximum area under the receiver operator characteristic curve (AUROC) was selected. The trained classifier was then used to obtain prediction scores on the hold-out test set. To evaluate robustness of the model training and to ensure complete cross-validation, the entire process was repeated seven times, resulting in thirty-five unique train and test splits. The performances obtained during each run were averaged, and the mean scores along with the 95% CI were reported. Features that were repeatedly chosen (>80% for MODS and >60% for individual organ dysfunctions) during multiple runs of the cross-validation experiments were determined. Classification performance of models in external validation cohorts were judged based on the AUROC and the Matthew's Correlation Coefficient (MCC)—a balanced statistical measure of true positive, true negative, false positive, and false negatives [27], as shown in FIG. 5.

The top (10,20,30) stable features chosen consistently across>80% of 35-fold cross-validation experiments were tested among the three external cohorts—adult (E-MTAB-5882) and pediatric (GSE144406, and E-MTAB-10938—the latter using both Proulx and PELOD definitions for MODS). A total of 210 top feature X classifier combinations were tested and the results for the top 5 MCC values for the external dataset are summarized in FIG. 5. Based on these results, the top 20 features and ET classifier were used as the final model.

### Parameter Tuning.

The validation E-MTAB10938 ArrayExpress dataset was used, published by Snyder et al. and consisting of 32 pediatric patients with septic shock, of whom 19 had an immunoparalysis phenotype of MODS [19] for parameter tuning. Briefly, the training used different feature sets (of sizes 5,10,15, . . . 111) identified through the training cohort, tuned the following parameters using the validation dataset: (1) optimal number of features, 2) sampling technique-classifier combination, and 3) optimal probability threshold for imbalanced classification.

### Testing Model Performance.

The performance of the final model was tested in two test datasets: GSE144406 GEO dataset published by Shankar et al. that consisted of a whole blood bulk RNA sequencing total of 27 pediatric patients including four healthy controls, 17 patients with MODS, and six patients with MODS requiring extracorporeal membrane oxygen (ECMO) support [17] and E-MTAB-5882 ArrayExpress dataset published by Cabrera et al that consisted of time-course-based gene-expression profiling measurements collected from the whole blood of 70 critically injured adult patients in the hyper-acute time period within 2 hours of trauma [26]. Classification performance of models in the validation and test sets were judged based on the AUROC and the Matthew's Correlation Coefficient (MCC)—a balanced statistical measure of true positive, true negative, false positive, and false negatives [27]. Model performance at a fixed sensitivity of 85% was reported across the validation and test cohorts. The 95% CI for each classification metric was derived by repeated sampling with replacement with 1000 iterations. The ci function from the gmodels package in R was used to calculate the CIs.

Four external datasets were used: 1) E-MTAB-5882 ArrayExpress dataset that consisted of time-course-based gene-expression profiling measurements collected from the whole blood of 70 critically injured adult patients in the hyperacute time period within 2 hours of trauma [26]; 2) E-MTAB-1548 ArrayExpress dataset comprised of 155 adult post-surgical patients with and without septic shock admitted to a Spanish ICU [43,44]; 3) GSE144406 GEO dataset that consisted of a whole blood bulk RNA sequencing total of 27 pediatric patients including 4 healthy controls, 17 patients with MODS, and 6 patients with MODS requiring extracorporeal membrane oxygen (ECMO) support [17]; and 4) E-MTAB-10938 ArrayExpress dataset that consisted of 32 pediatric patients with septic shock, of whom 19 had an immunoparalysis phenotype of MODS [19]. Similar, quality control measures were used during data pre-processing of validation cohorts as with the derivation cohort. Different combinations of top genes (n=10, 20, . . . 50) correlated with MODS in the derivation cohort were tested with numerous classifier and sampling techniques to estimate risk of MODS in validation cohorts. The minimal number of genes was then determined, along with a single classifier combination that provided consistent performance across validation cohorts. Model performances at a fixed sensitivity of 85% [45] were reported across validation cohorts.

### Performance Relative to Established Genes Predictive of Sepsis Mortality.

The study included a determination of whether genes identified through the supervised ML model were comparable or improved upon published literature on gene sets, which have been demonstrated to predict sepsis mortality, in identifying at patients with persistent MODS. A total of 58 genes were outlined in Sweeney et al. that were predictive of 30-day mortality [12]. However, only 51/58 genes were present among training, validation, and test datasets and were chosen for further analysis. The same optimization was followed as with the test sets but using 51 genes predictive of mortality instead of those predictive of MODS.

### Organ-Specific Dysfunction Signatures.

Gene signatures were determined that correlated with three major organ dysfunctions cardiovascular, respiratory, and kidney dysfunction—at day 3 and day 7 time points independently in the derivation cohort. Based on the presumption that the MODS signature represented the shared biological pathways among patients with ≥2 of cardiovascular, respiratory, renal, hepatic, hematologic, and neurologic dysfunctions, organ-specific differentially expressed genes were identified by eliminating redundancies associated with the shared MODS signature. Finally, targets correlated with cardiovascular, respiratory, and renal dysfunction were identified, which feature in ≥60% of cross-validation experiments.

### Endotype Identification.

Unsupervised hierarchical clustering of the top genes correlated with persistent MODS, selected based on best stability score [42], were used to derive patient subclasses within the derivation cohort. Clinical relevance of newly derived subclasses was determined by estimating differences in clinical outcomes, organ support, and response to adjuvant corticosteroid therapy. Finally, comparisons between previously validated septic shock endotypes [15](endotype A and endotype B) available in patients with septic shock and the newly derived MODS subclasses were made. Reactome pathway analyses was used to the determine implicated biological processes [41]. Differences in 50 genes used to determine patient subclasses were compared between MODS endotypes.

### Statistical Analysis.

Demographic and clinical data were summarized with counts and percentages or medians with interquartile ranges (IQR). Differences between groups were determined by x2 test for categorical variables and by one-way analysis of variance (ANOVA) for continuous variables. A p-value of 0.05 was used to test statistical significance, unless otherwise specified.

Dunn's test was used for account for multiple comparisons testing, where applicable. Log rank test was used to compare 28-day survival among patient subclasses. Logistic regression analyses were used to test the association between MODS endotypes, receipt of adjuvant steroids, and 28-day mortality and occurrence of MODS. All models included the interaction variable between endotype X receipt of steroids.

### Example 4

### Patient Characteristics

A total of 201 patients with phenotyping of multiple organ dysfunction trajectories were included in the training dataset. The demographic characteristics of the cohort are shown in Table 3. Forty-six patients had persistent MODS, including 15 patients who died within 7 days of study enrollment. Sixty-three patients had resolving MODS. Those with no MODS included 19 patients with sepsis without shock or organ dysfunctions on day 1, 26 patients with SIRS, and 47 patients admitted for elective surgical procedures who served as healthy controls.

Patients with persistent MODS were younger, had higher illness severity at baseline, and a trend toward higher day 1 vasoactive inotropic scores (VIS). There were no significant differences in rate of prescribed corticosteroids between groups. Unsurprisingly, those with persistent MODS had significantly higher 28-day mortality, fewer PICU free days, and higher cardiovascular, respiratory, and renal support requirements than those with resolving or no MODS. Individual organ dysfunctions and supportive interventions by MODS trajectory are detailed by day of septic shock in Table 4 and Table 5, respectively.

TABLE 3
Demographic and outcome data by MODS trajectory in derivation cohort.

| | Persistent MODS | | Resolving MODS | | Other Controls | | P Value |
|---|---|---|---|---|---|---|---|
| N (%) | 46 | (22.7%) | 63 | (31.0%) | 92 | (46.3%) | |
| Age (years) | 1.8 | (0.5, 4.5) | 2.4 | (1.1, 5.2) | 2.9 | (1.3, 6.1) | 0.03 |
| Sex, m | 28 | (60.8%) | 35 | (55.5%) | 50 | (53.2%) | 0.69 |
| Race | | | | | | | |
| White | 29 | | 40 | | N/A | | 0.83 |
| Black | 11 | | 18 | | | | |
| Other | 6 | | 5 | | | | |
| PRISM-III | 21 | (15, 29) | 14 | (10, 18) | 1 | (0, 10) | 0.01* |
| Day 1 VIS score | 20 | (1, 55) | 10 | (1, 20) | 0 | (0, 0) | 0.07 |
| Source | | | | | | | |
| Pulmonary | 9 | | 7 | | 4 | | 0.45* |
| Extrapulmonary | 23 | | 28 | | 7 | | |
| None | 14 | | 28 | | 83 | | |
| Pathogen type | | | | | | | 0.66* |
| Gram positive | 19 | | 15 | | 5 | | |
| Gram negative | 10 | | 15 | | 6 | | |
| Viral | 2 | | 4 | | 0 | | |
| Fungal | 1 | | 1 | | 0 | | |
| Outcomes | | | | | | | |
| 28-day mortality | 17 | | 1 | | 0 | | <0.01 |
| PICU free days | 12 | (0, 19) | 22 | (17, 24) | 23 | (19, 25) | <0.01 |
| PICU LOS | 10 | (3, 19) | 6 | (4, 11) | 5 | (3, 8) | 0.02 |
| Hospital LOS | 19 | (3, 33) | 10 | (8, 21) | 9 | (7, 14) | 0.38 |
| Steroid use | 18 | (39.2%) | 15 | (30.6%) | 4 | (4.4%) | 0.38 |

PRISM III: Pediatric Risk of Mortality III score; VIS score: Vasoactive inotropic score; LOS: Length of stay; N/A: Not available.

TABLE 4
Organ dysfunctions by MODS trajectory on day 1, 3, and
7 of septic shock diagnosis in the training dataset.

| | Evolving MODS | Resolving MODS | P Value |
|---|---|---|---|
| Day 1 MODS | N = 44 | N = 62 | 0.74 |
| Cardiovascular | 43 | 57 | 0.57 |
| Respiratory | 44 | 45 | 0.67 |
| Renal | 36 | 11 | <0.01 |
| Hepatic | 22 | 5 | <0.01 |
| Hematologic | 33 | 13 | <0.01 |
| Neurologic | 9 | 0 | <0.01 |
| Day 3 MODS | N = 44 | N = 26 | <0.01 |
| Cardiovascular | 38 | 32 | <0.01 |
| Respiratory | 43 | 29 | <0.01 |
| Renal | 36 | 8 | <0.01 |
| Hepatic | 23 | 4 | <0.01 |
| Hematologic | 28 | 12 | <0.01 |
| Neurologic | 14 | 0 | <0.01 |
| Day 7 MODS | N = 46 | N = 0 | <0.01 |
| Cardiovascular | 32 | 3 | <0.01 |
| Respiratory | 42 | 7 | <0.01 |
| Renal | 34 | 4 | <0.01 |
| Hepatic | 23 | 1 | <0.01 |
| Hematologic | 24 | 1 | <0.01 |
| Neurologic | 14 | 0 | <0.01 |

TABLE 5
Organ support on day 1, 3, and 7 of septic shock diagnosis.

| | Evolving MODS | Resolving MODS | P Value |
|---|---|---|---|
| Day 1 MODS | N = 44 | N = 63 | 0.74 |
| Vasoactive support | 39 | 45 | 0.26 |
| Ventilatory support | 41 | 35 | 0.03 |
| Renal replacement | 13 | 1 | <0.01 |
| Day 3 MODS | N = 44 | N = 26 | <0.01 |
| Vasoactive support | 37 | 32 | <0.01 |
| Ventilatory support | 43 | 29 | <0.01 |
| Renal replacement | 21 | 1 | <0.01 |
| Day 7 MODS | N = 46 | N = 0 | <0.01 |
| Vasoactive support | 32 | 4 | <0.01 |
| Ventilatory support | 42 | 8 | <0.01 |
| Renal replacement | 22 | 1 | <0.01 |

### Example 5

### Gene Expression Signature Associated with Persistent MODS Trajectory and its Biological Relevance

568 genes were found to be differentially expressed among patients with persistent MODS relative to those with resolving or no MODS; 369 genes were upregulated, and 199 genes were downregulated. The heat map and volcano plot for DEG analyses are shown in FIG. 5.

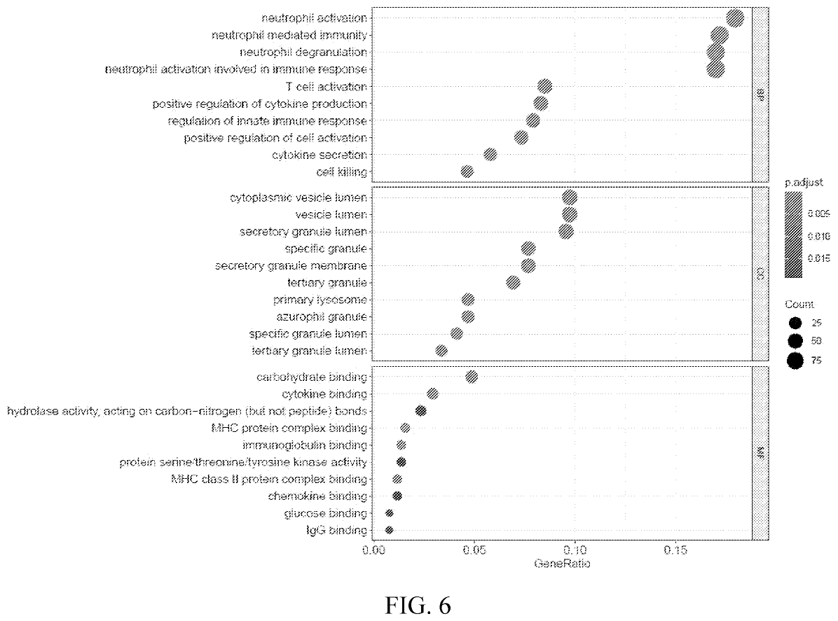

In sensitivity analyses, exclusion of patients who died within the first 7 days (n=15) did not significantly alter the results. This analysis identified 111 genes consistently associated with persistent MODS on repeated cross-validation experiments, detailed in Table 6. The top 10 genes identified were RETN, ADAMTS3, LDHA, LCN2, IL1R2, DDIT4, CEACAM8, MERTK, MPO, and ARL4A. Biological pathways enriched among those with persistent MODS included: “neutrophil degranulation”, “immune system”, “innate immune system”, “adaptive immune system” and “cytokine signaling in the immune system”, as shown in FIG. 6.

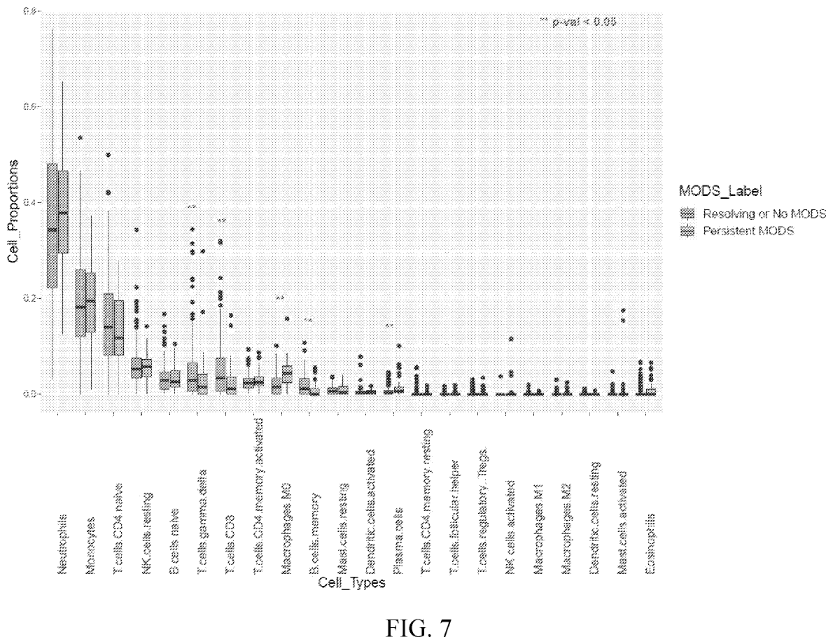

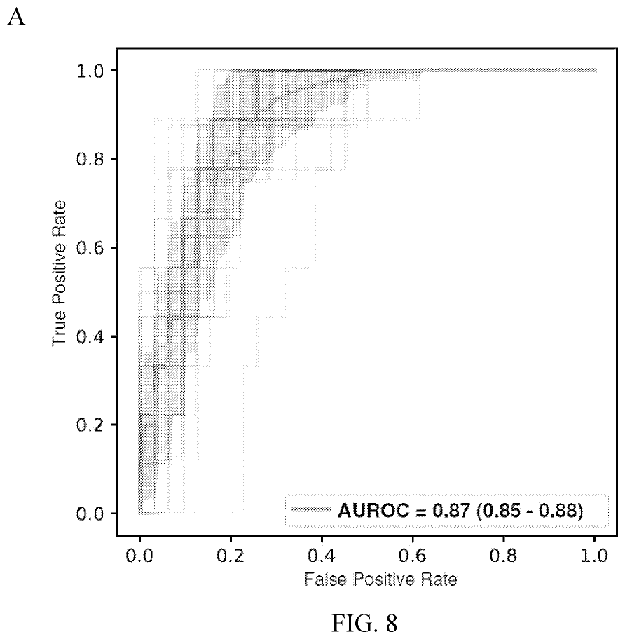

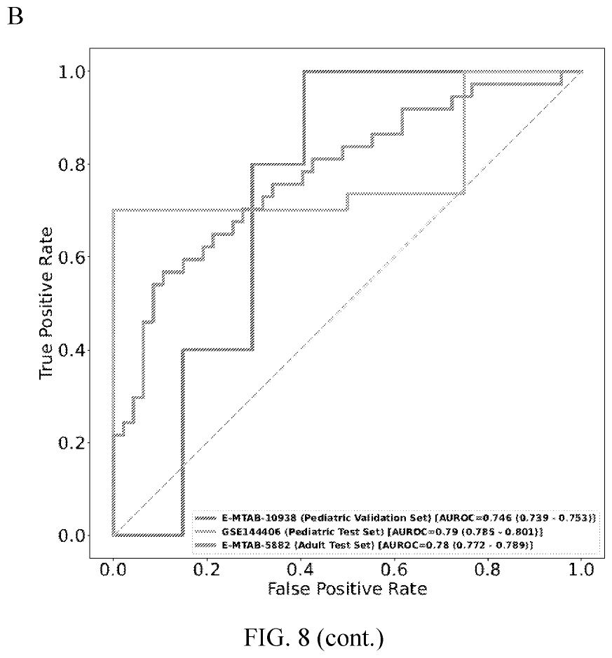

CIBERSORT analyses revealed that although neutrophils and monocytes accounted for the most abundant cell types, there were no significant differences among estimated cell proportions among those with persistent MODS relative to those without. However, an overrepresentation of M0 macrophages and plasma cells and an under-representation of CD8+ T cells, Naive CD4+ T cells, γδ T cells, and memory B cells was observed among patients with persistent MODS relative to those without, as shown in FIG. 7 and detailed in Table 7.

Results of propensity matching are detailed in Table 8. The propensity score for each patient was consistently among the top features identified by these ML models and strengthened the performance of the risk prediction model. The method identified 109 genes consistently correlated with persistent MODS in ≥80% of cross-validation experiments, detailed in Table 9. The top 10 genes identified were RETN, ADAMTS3, LDHA, LCN2, IL1R2, DDIT4, CEACAM8, MERTK, MPO, and ARL4A.

Where available, inclusion of propensity scores enhanced model performance to estimate risk of MODS in the validation cohorts, as shown in Table 10.

TABLE 6
Top genes correlated with persistent MODS and identified
in >80% of cross-validation experiments.

| # | GENE | FRACTION |
|---|---|---|
| 1 | RETN | 1 |
| 2 | ADAMTS3 | 1 |
| 3 | LDHA | 1 |
| 4 | LCN2 | 1 |
| 5 | IL1R2 | 0.971 |
| 6 | DDIT4 | 0.971 |
| 7 | CEACAM8 | 0.971 |
| 8 | MERTK | 0.971 |
| 9 | MPO | 0.971 |
| 10 | ARL4A | 0.971 |
| 11 | CDKN3 | 0.971 |
| 12 | PRTN3 | 0.971 |
| 13 | ID1 | 0.971 |
| 14 | MTMR11 | 0.971 |
| 15 | ANLN | 0.971 |
| 16 | KIF20A | 0.971 |
| 17 | IL1RAP | 0.943 |
| 18 | HLA-DMB | 0.943 |
| 19 | RAB13 | 0.943 |
| 20 | ZBTB16 | 0.943 |
| 21 | NUSAP1 | 0.943 |
| 22 | GGH | 0.943 |
| 23 | MMP8 | 0.943 |
| 24 | TRBV27 | 0.943 |
| 25 | PRC1 | 0.943 |
| 26 | COX6C | 0.943 |
| 27 | CD24 | 0.943 |
| 28 | CTSL | 0.943 |
| 29 | A2M-AS1 | 0.914 |
| 30 | MAFF | 0.914 |
| 31 | TMEM272 | 0.914 |
| 32 | NFE2 | 0.914 |
| 33 | BLM | 0.914 |
| 34 | OLFM4 | 0.914 |
| 35 | MAP3K7CL | 0.914 |
| 36 | CEACAM6 | 0.886 |
| 37 | FCER1A | 0.886 |
| 38 | CEP55 | 0.886 |
| 39 | TLR7 | 0.886 |
| 40 | GPI | 0.886 |
| 41 | SLC46A2 | 0.886 |
| 42 | FCGR2B | 0.886 |
| 43 | SLC51A | 0.886 |
| 44 | H1-2 | 0.886 |
| 45 | PNPLA6 | 0.886 |
| 46 | LTF | 0.886 |
| 47 | HLA-DPA1 | 0.886 |
| 48 | MS4A4A | 0.886 |
| 49 | CENPW | 0.886 |
| 50 | FGFBP2 | 0.886 |
| 51 | CEACAM1 | 0.886 |
| 52 | TAGAP | 0.886 |
| 53 | PRG2 | 0.886 |
| 54 | DAAM2 | 0.857 |
| 55 | ORM1 | 0.857 |
| 56 | IFI44L | 0.857 |
| 57 | SLCO4A1 | 0.857 |
| 58 | BEX1 | 0.857 |
| 59 | AATBC | 0.857 |
| 60 | IFIT1 | 0.857 |
| 61 | NELL2 | 0.857 |
| 62 | RPS6KA5 | 0.857 |
| 63 | C1QB | 0.857 |
| 64 | COL17A1 | 0.857 |
| 65 | PARP8 | 0.857 |
| 66 | CX3CR1 | 0.857 |
| 67 | TBC1D4 | 0.857 |
| 68 | TOP2A | 0.857 |
| 69 | HSP90AA1 | 0.857 |
| 70 | TCEAL9 | 0.857 |
| 71 | ARG1 | 0.829 |
| 72 | SUCNR1 | 0.829 |
| 73 | KIF14 | 0.829 |
| 74 | RHAG | 0.829 |
| 75 | TGFBI | 0.829 |
| 76 | OLAH | 0.829 |
| 77 | CRTAM | 0.829 |
| 78 | CR1L | 0.829 |
| 79 | ETS2 | 0.829 |
| 80 | CYSTM1 | 0.829 |
| 81 | TUBG1 | 0.829 |
| 82 | UHRF1 | 0.829 |
| 83 | CTSG | 0.829 |
| 84 | HGF | 0.829 |
| 85 | NDUFA1 | 0.829 |
| 86 | ZNF600 | 0.829 |
| 87 | TMEM45A | 0.829 |
| 88 | MYL6B | 0.829 |
| 89 | C15orf65 | 0.829 |
| 90 | RASGRP1 | 0.829 |
| 91 | PTX3 | 0.829 |
| 92 | HIPK2 | 0.829 |
| 93 | CD86 | 0.8 |
| 94 | ELANE | 0.8 |
| 95 | LY9 | 0.8 |
| 96 | THBS1 | 0.8 |
| 97 | NR3C2 | 0.8 |
| 98 | NARF | 0.8 |
| 99 | HCAR3 | 0.8 |
| 100 | CFD | 0.8 |
| 101 | IL10RB-DT | 0.8 |
| 102 | CCNE2 | 0.8 |
| 103 | IFIT5 | 0.8 |
| 104 | CLEC4D | 0.8 |
| 105 | GADD45A | 0.8 |
| 106 | C15orf48 | 0.8 |
| 107 | ROMO1 | 0.8 |
| 108 | PADI4 | 0.8 |
| 109 | NUF2 | 0.8 |

Fraction: represents % of cross-validation experiments in which the gene was associated with persistent MODS in the derivation cohort.

TABLE 7
CIBERSORT analyses of cell type abundance between
patients with evolving MODS and those without.

| | Evolving | No Evolving | |
|---|---|---|---|
| Cell Type | MODS | MODS | Significance |
| Neutrophils | 0.377 | 0.343 | 0.115 |
| Monocytes | 0.179 | 0.176 | 0.601 |
| T cells Cd4 naïve | 0.132 | 0.138 | 0.048 ** |
| NK cells resting | 0.055 | 0.052 | 0.419 |
| Macrophages M0 | 0.039 | 0.015 | 0.000 ** |
| B cells naïve | 0.027 | 0.028 | 0.934 |
| T cells Cd4 memory | 0.025 | 0.022 | 0.181 |
| activated | | | |
| T cells Cd8 | 0.019 | 0.032 | 0.006 ** |
| T cells gamma delta | 0.013 | 0.03 | 0.019 ** |
| Mast cells resting | 0.008 | 0.007 | 0.528 |
| Plasma cells | 0.006 | 0.002 | 0.014 ** |
| Dendritic cells activated | 0.004 | 0.003 | 0.944 |
| Dendritic cells resting | 0 | 0 | 0.038 |
| B cells memory | 0 | 0.01 | 0.072 |

** Statistically significant differences in cell type abundance between patients with evolving and no evolving MODS.

TABLE 8
Results of propensity matching for age and illness severity.

| | | | Standard Mean |
|---|---|---|---|
| | Mean Treated | Mean Control | Difference |
| Before Matching | | | |
| Distance | 0.36 | 0.13 | 0.91 |
| Age (years) | 3.11 | 3.73 | −0.22 |
| Prism iii | 18.8 | 8.3 | 1.14 |
| After Matching | | | |
| Distance | 0.35 | 0.33 | 0.08 |
| Age (years) | 3.11 | 3.19 | −0.02 |
| Prism iii | 18.7 | 17.8 | 0.09 |

TABLE 9
Top features correlated with evolving MODS and identified
in >80% of cross-validation experiments.

| # | GENE | FRACTION |
|---|---|---|
| 1 | RETN | 1 |
| 2 | ADAMTS3 | 1 |
| 3 | LDHA | 1 |
| 4 | Propensity | 1 |
| 5 | LCN2 | 1 |
| 6 | IL1R2 | 0.971 |
| 7 | DDIT4 | 0.971 |
| 8 | CEACAM8 | 0.971 |
| 9 | MERTK | 0.971 |
| 10 | MPO | 0.971 |
| 11 | ARL4A | 0.971 |
| 12 | CDKN3 | 0.971 |
| 13 | PRTN3 | 0.971 |
| 14 | ID1 | 0.971 |
| 15 | MTMR11 | 0.971 |
| 16 | ANLN | 0.971 |
| 17 | KIF20A | 0.971 |
| 18 | IL1RAP | 0.943 |
| 19 | HLA-DMB | 0.943 |
| 20 | RAB13 | 0.943 |
| 21 | ZBTB16 | 0.943 |
| 22 | NUSAP1 | 0.943 |
| 23 | GGH | 0.943 |
| 24 | MMP8 | 0.943 |
| 25 | TRBV27 | 0.943 |
| 26 | PRC1 | 0.943 |
| 27 | COX6C | 0.943 |
| 28 | CD24 | 0.943 |
| 29 | CTSL | 0.943 |
| 30 | A2M-AS1 | 0.914 |
| 31 | MAFF | 0.914 |
| 32 | TMEM272 | 0.914 |
| 33 | NFE2 | 0.914 |
| 34 | BLM | 0.914 |
| 35 | OLFM4 | 0.914 |
| 36 | MAP3K7CL | 0.914 |
| 37 | CEACAM6 | 0.886 |
| 38 | FCER1A | 0.886 |
| 39 | CEP55 | 0.886 |
| 40 | TLR7 | 0.886 |
| 41 | GPI | 0.886 |
| 42 | SLC46A2 | 0.886 |
| 43 | FCGR2B | 0.886 |
| 44 | SLC51A | 0.886 |
| 45 | H1-2 | 0.886 |
| 46 | PNPLA6 | 0.886 |
| 47 | LTF | 0.886 |
| 48 | HLA-DPA1 | 0.886 |
| 49 | MS4A4A | 0.886 |
| 50 | CENPW | 0.886 |
| 51 | FGFBP2 | 0.886 |
| 52 | CEACAM1 | 0.886 |
| 53 | TAGAP | 0.886 |
| 54 | PRG2 | 0.886 |
| 55 | DAAM2 | 0.857 |
| 56 | ORM1 | 0.857 |
| 57 | IFI44L | 0.857 |
| 58 | SLCO4A1 | 0.857 |
| 59 | BEX1 | 0.857 |
| 60 | AATBC | 0.857 |
| 61 | IFIT1 | 0.857 |
| 62 | NELL2 | 0.857 |
| 63 | RPS6KA5 | 0.857 |
| 64 | C1QB | 0.857 |
| 65 | COL17A1 | 0.857 |
| 66 | PARP8 | 0.857 |
| 67 | CX3CR1 | 0.857 |
| 68 | TBC1D4 | 0.857 |
| 69 | TOP2A | 0.857 |
| 70 | HSP90AA1 | 0.857 |
| 71 | TCEAL9 | 0.857 |
| 72 | ARG1 | 0.829 |
| 73 | SUCNR1 | 0.829 |
| 74 | KIF14 | 0.829 |
| 75 | RHAG | 0.829 |
| 76 | TGFBI | 0.829 |
| 77 | OLAH | 0.829 |
| 78 | CRTAM | 0.829 |
| 79 | CR1L | 0.829 |
| 80 | ETS2 | 0.829 |
| 81 | CYSTM1 | 0.829 |
| 82 | TUBG1 | 0.829 |
| 83 | UHRF1 | 0.829 |
| 84 | CTSG | 0.829 |
| 85 | HGF | 0.829 |
| 86 | NDUFA1 | 0.829 |
| 87 | ZNF600 | 0.829 |
| 88 | TMEM45A | 0.829 |
| 89 | MYL6B | 0.829 |
| 90 | C15orf65 | 0.829 |
| 91 | RASGRP1 | 0.829 |
| 92 | PTX3 | 0.829 |
| 93 | HIPK2 | 0.829 |
| 94 | CD86 | 0.8 |
| 95 | ELANE | 0.8 |
| 96 | LY9 | 0.8 |
| 97 | THBS1 | 0.8 |
| 98 | NR3C2 | 0.8 |
| 99 | NARF | 0.8 |
| 100 | HCAR3 | 0.8 |
| 101 | CFD | 0.8 |
| 102 | IL10RB-DT | 0.8 |
| 103 | CCNE2 | 0.8 |
| 104 | IFIT5 | 0.8 |
| 105 | CLEC4D | 0.8 |
| 106 | GADD45A | 0.8 |
| 107 | C15orf48 | 0.8 |
| 108 | ROMO1 | 0.8 |
| 109 | PADI4 | 0.8 |
| 110 | NUF2 | 0.8 |

Fraction: represents % of cross-validation experiments in which the respective feature was correlated with evolving MODS in the derivation cohort.

TABLE 10
Model performance in external validation datasets.
Top 20 genes with ExtraTree Classifier

| Age group | Dataset | AUROC | Sensitivity | Specificity | PPV |
|---|---|---|---|---|---|
| Pediatric | GSE166640 | 0.96 | 0.85 | 1 | 1 |
| Pediatric | E-MTAB-10938 | 0.78 | 0.85 | 0.72 | 0.37 |
| Adult | E-MTAB-1548 | 0.82 | 0.85 | 0.58 | 0.5 |
| Adult | E-MTAB-5882 | 0.77 | 0.85 | 0.44 | 0.53 |

Top 20 genes with ExtraTree Classifier + Propensity Score

| Age group | Dataset | AUROC | Sensitivity | Specificity | PPV |
|---|---|---|---|---|---|
| Pediatric | GSE166640 | Not applicable, dataset lacked an illness severity score. | | | |
| Pediatric | E-MTAB-10938 | 0.8 | 0.85 | 0.78 | 0.4 |
| Adult | E-MTAB-1548 | 0.84 | 0.85 | 0.56 | 0.49 |
| Adult | E-MTAB-5882 | 0.83 | 0.85 | 0.49 | 0.59 |

AUROC: Area under the receiver operator characteristic; PPV: Positive predictive value

### Example 6

### Genes Associated with Persistent MODS can be Used to Reliably Identify Those at-Risk Among Children and Adults with Different Causes for Organ Dysfunction

The AUROC for the risk prediction model that included these 111 genes to distinguish patients with MODS relative to those with resolving or no MODS in the training dataset was 0.87 (95% CI: 0.85-0.88) with an MCC of 0.64 (95% CI: 0.60-0.68). The model had a sensitivity of 94.0% (87-93%) and specificity of 79% (76-83%).

In the validation dataset, the analysis identified that the optimal parameters to predict those at risk of MODS were achieved by using 20 out of 111 genes (specifically, RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8) identified in the training dataset, Instance Hardness Threshold (IHT) sampling technique, and Extra Trees (ET) classifier at a threshold of 0.488; this type of ensemble learning technique aggregates the results of multiple de-correlated decision trees collected in a forest to output it's classification result. The AUROC of the model to predict MODS in the validation cohort of pediatric septic shock patients with immunoanalysis phenotype was 0.74 (95% CI: 0.73-0.75). Finally, the AUROCs to predict MODS for the final model with fixed parameters were 0.79 (95% CI: 0.78-0.80) in the GSE144406 dataset of pediatric patients, some of whom received ECMO support and 0.78 (95% CI: 0.77-0.78) in E-MTAB-5882 among adults patients in the hyper-acute phase of trauma. Model performance in validation and test sets are summarized in Table 11. The AUROCs of models in the training, validation, and test sets are shown in FIG. 7.

TABLE 11
Model performance across validation and test sets using 20 gene predictive
of MODS and fixed parameters reported at a sensitivity of 85%.

| | | | | | | | | Balanced |
|---|---|---|---|---|---|---|---|---|
| Type | Dataset | Distribution | Sensitivity | Specificity | AUC | Precision | MCC | Accuracy |
| Validation | Pediatric Sepsis | #Positive: 7 | 0.85 | 0.70 | 0.75 | 0.33 | 0.36 | 0.75 |
| | Immunoparalysis | #Negative: 25 | | (0.70-0.71) | (0.74-0.75) | (0.32-0.34) | (0.35-0.38) | (0.73-0.77) |
| | (E-MTAB-10938) | | | | | | | |
| Test | Pediatric Sepsis | #Positive: 4 | 0.85 | 0.30 | 0.79 | 0.94 | 0.13 | 0.6 |
| | ECMO vs. not on | #Negative: 57 | | (0.29-0.31) | (0.78-0.80) | (0.94-0.94) | (0.12-0.15) | (0.59-0.61) |
| | ECMO. | | | | | | | |
| | (GSE144406) | | | | | | | |
| Test | Adult Hyperacute | #Positive: 37 | 0.85 | 0.51 | 0.78 | 0.58 | 0.36 | 0.67 |
| | phase of trauma | #Negative: 47 | | (0.50-0.51) | (0.77-0.79) | (0.57-0.58) | (0.36-0.36) | (0.69-0.67) |
| | (E-MTAB-5882) | | | | | | | |

Model parameters included top 20 genes, Standard Scaler, Instance Hardness Threshold sampling technique, and Extra-trees Classifier at a threshold of 0.488.

### Example 7

### Model Performance Compared to Published Gene-Sets Predictive of Sepsis Mortality to Predict Risk of MODS

51 genes predictive of sepsis mortality with comparable ML model parameters including Extra Trees classifier model, MinMax Scaler, CC sampler, and a threshold of 0.429 were used to predict MODS in the validation and test datasets. Although this gene-set demonstrated a comparable AUROC in the validation set E-MTAB-10938 model performance in the test sets varied widely, with AUROCs ranging from 0.56-0.76. These results are shown in Table 12.

TABLE 12
Model performance to estimate risk of MODS across validation and test sets using 51 gene
predictive of sepsis mortality and fixed parameters reported at a sensitivity of 85%.

| | | | | | | | | Balanced |
|---|---|---|---|---|---|---|---|---|
| Type | Dataset | Distribution | Sensitivity | Specificity | AUC | Precision | MCC | Accuracy |
| Validation | Pediatric Sepsis | #Positive: 7 | 0.85 | 0.7 | 0.76 | 0.33 | 0.37 | 0.75 |
| | Immunoparalysis | #Negative: 25 | | (0.69-0.70) | (0.75-0.76) | (0.3-0.34) | (0.37-0.38) | (0.75-0.76) |
| | (E-MTAB-10938) | | | | | | | |
| Test | Pediatric Sepsis | #Positive: 4 | 0.85 | 0.0 | 0.57 | 0.92 | −0.1 | 0.43 |
| | ECMO vs. not on | #Negative: 57 | | | (0.56-0.57) | (0.91-0.92) | (−0.15-(−0.08)) | (0.43-0.43) |
| | ECMO. | | | | | | | |
| | (GSE144406) | | | | | | | |
| Test | Adult Hyperacute | #Positive: 37 | 0.85 | 0.34 | 0.76 | 0.507 | 0.23 | 0.60 |
| | phase of trauma | #Negative: 47 | | (0.33-0.34) | (0.75-0.77) | (0.50-0.51) | (0.22-0.24) | (0.60-0.61) |
| | (E-MTAB-5882) | | | | | | | |

Model parameters included 51 genes predictive of sepsis mortality, MinMaxScaler, CC sampling technique, and Extra-trees Classifier at a threshold of 0.429.

### Example 8

### Eliminating Redundancies to Disentangle Organ-Specific Dysfunction Signatures

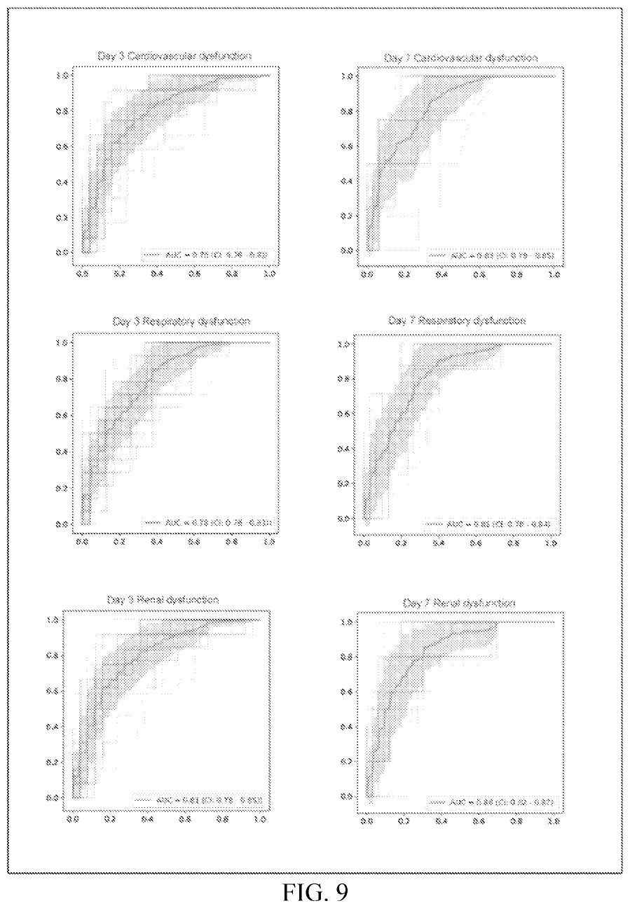

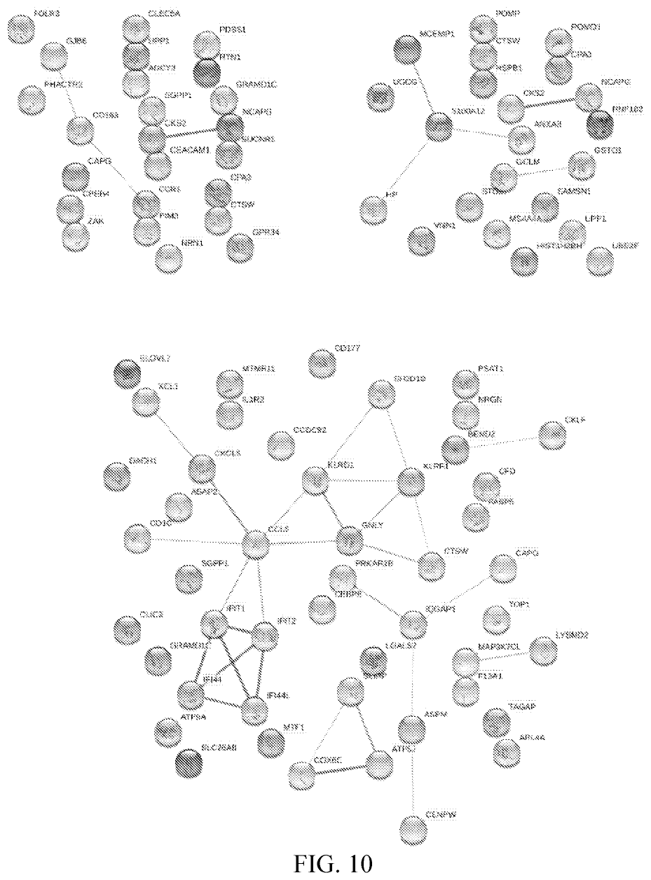

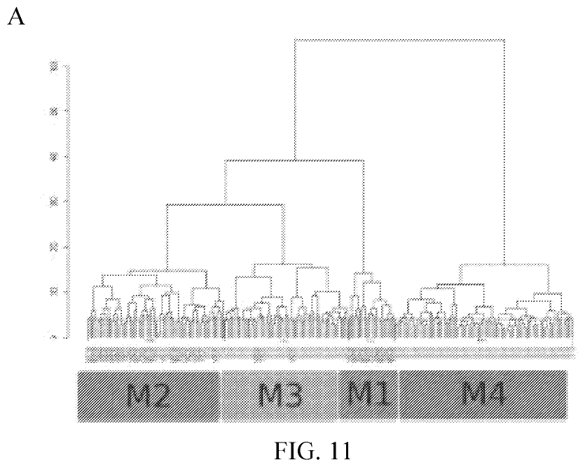

Top features associated with each of the three major organ dysfunctions—cardiovascular, respiratory, and kidney—were identified independently at day 3 and 7, detailed in Tables 13-18. The AUROC for organ-specific models at both time points are summarized in FIG. 9. By eliminating redundancies associated with the shared MODS signature, genes correlated with individual organ dysfunctions were determined, as detailed in Tables 19-24. CCR1, GPR34, and PDSS1 were the top 3 transcripts correlated with day 3 cardiovascular dysfunction; HSPB1, LILRA3, and GSTO1 were the top 3 transcripts correlated with day 3 respiratory dysfunction; CXCL5, CKLF, and NRGN were the genes correlated with day 3 renal dysfunction. The relevant protein-protein interactions for day 3 organ dysfunctions are shown in FIG. 10.

TABLE 13
Top features correlated with day 3 cardiovascular dysfunction
in > 60% of cross-validation experiments.

| # | GENE | FRACTION |
|---|---|---|
| 1 | CPEB4 | 1 |
| 2 | MIAT | 1 |
| 3 | PTX3 | 0.971 |
| 4 | CLEC5A | 0.971 |
| 5 | GPR34 | 0.971 |
| 6 | CCR1 | 0.971 |
| 7 | ANLN | 0.943 |
| 8 | PLCB1 | 0.914 |
| 9 | PDSS1 | 0.914 |
| 10 | LINC02850 | 0.914 |
| 11 | EML5 | 0.886 |
| 12 | LILRA3 | 0.886 |
| 13 | CREM | 0.886 |
| 14 | NRN1 | 0.857 |
| 15 | CEACAM8 | 0.857 |
| 16 | TARS1 | 0.857 |
| 17 | PIM3 | 0.857 |
| 18 | PHACTR2 | 0.829 |
| 19 | SGPP1 | 0.829 |
| 20 | CEACAM1 | 0.829 |
| 21 | CPA3 | 0.829 |
| 22 | RTN1 | 0.829 |
| 23 | GPBAR1 | 0.829 |
| 24 | FOLR3 | 0.829 |
| 25 | PLCD1 | 0.829 |
| 26 | TOP2A | 0.8 |
| 27 | PLCG2 | 0.771 |
| 28 | GRAMD1C | 0.771 |
| 29 | LINC00597 | 0.771 |
| 30 | OLFM4 | 0.771 |
| 31 | OLAH | 0.743 |
| 32 | LOC114224 | 0.743 |
| 33 | ADCY3 | 0.743 |
| 34 | CAPG | 0.743 |
| 35 | EIF4G3 | 0.743 |
| 36 | SUCNR1 | 0.743 |
| 37 | MS4A3 | 0.714 |
| 38 | CD163 | 0.714 |
| 39 | ABCA1 | 0.714 |
| 40 | CTSW | 0.714 |
| 41 | PROK2 | 0.714 |
| 42 | LINC01000 | 0.714 |
| 43 | MAP3K20 | 0.714 |
| 44 | PLCB4 | 0.714 |
| 45 | ELANE | 0.714 |
| 46 | LINC00102 | 0.714 |
| 47 | TTK | 0.714 |
| 48 | MICOS10P1 | 0.686 |
| 49 | PEAK1 | 0.686 |
| 50 | PRC1 | 0.686 |
| 51 | GSEC | 0.657 |
| 52 | CKS2 | 0.657 |
| 53 | NCAPG | 0.657 |
| 54 | GJB6 | 0.657 |
| 55 | SHOC1 | 0.657 |
| 56 | UPP1 | 0.657 |
| 57 | Propensity | 0.657 |
| 58 | TCN1 | 0.657 |
| 59 | CEP55 | 0.657 |
| 60 | CD177 | 0.629 |
| 61 | IL10RB-DT | 0.629 |
| 62 | CD160 | 0.629 |
| 63 | SHCBP1 | 0.629 |
| 64 | SOCS3 | 0.629 |
| 65 | PLIN3 | 0.629 |
| 66 | UHRF1 | 0.629 |
| 67 | ASAP1-IT1 | 0.629 |
| 68 | SFXN1 | 0.629 |
| 69 | KIF14 | 0.6 |
| 70 | CENPW | 0.6 |
| 71 | LGALS1 | 0.6 |
| 72 | XCL1 | 0.6 |
| 73 | LINC01943 | 0.6 |
| 74 | SULF2 | 0.6 |

TABLE 14
Top features correlated with day 3 respiratory dysfunction
in > 60% of cross-validation experiments.

| # | GENE | FRACTION |
|---|---|---|
| 1 | VNN1 | 0.943 |
| 2 | Propensity | 0.943 |
| 3 | STOM | 0.886 |
| 4 | GCLM | 0.886 |
| 5 | UGCG | 0.886 |
| 6 | ROMO1 | 0.857 |
| 7 | HSPB1 | 0.857 |
| 8 | LILRA3 | 0.857 |
| 9 | CPA3 | 0.857 |
| 10 | GSTO1 | 0.829 |
| 11 | CEACAM8 | 0.829 |
| 12 | PLAU | 0.8 |
| 13 | CKS2 | 0.771 |
| 14 | NCAPG | 0.771 |
| 15 | UPP1 | 0.771 |
| 16 | PTX3 | 0.771 |
| 17 | OLFM4 | 0.743 |
| 18 | RNF182 | 0.743 |
| 19 | UBE2F | 0.743 |
| 20 | H2BS1 | 0.743 |
| 21 | PSAT1 | 0.743 |
| 22 | CD24 | 0.714 |
| 23 | PLCB1 | 0.714 |
| 24 | CTSW | 0.714 |
| 25 | POMP | 0.714 |
| 26 | ANLN | 0.714 |
| 27 | S100A12 | 0.714 |
| 28 | MCEMP1 | 0.686 |
| 29 | PDZK1IP1 | 0.686 |
| 30 | TGFBI | 0.686 |
| 31 | SAMSN1 | 0.686 |
| 32 | AZU1 | 0.657 |
| 33 | ELANE | 0.657 |
| 34 | ANXA3 | 0.657 |
| 35 | MS4A4A | 0.657 |
| 36 | RETN | 0.657 |
| 37 | C2orf15 | 0.657 |
| 38 | PLCB4 | 0.657 |
| 39 | HP | 0.657 |
| 40 | LCN2 | 0.657 |
| 41 | H2BC9 | 0.657 |
| 42 | NOP10 | 0.629 |
| 43 | RAB10 | 0.629 |
| 44 | ECRP | 0.629 |
| 45 | CRISP3 | 0.629 |
| 46 | LTF | 0.629 |
| 47 | FCER1A | 0.629 |
| 48 | FCER1G | 0.629 |
| 49 | CEACAM1 | 0.629 |
| 50 | ATP2B1- | 0.629 |
| | AS1 | |
| 51 | DDAH2 | 0.6 |
| 52 | RPS6KA5 | 0.6 |
| 53 | GCA | 0.6 |
| 54 | ATP5PF | 0.6 |
| 55 | H2BC5 | 0.6 |
| 56 | PLCD1 | 0.6 |

TABLE 15
Top features correlated with day 3 renal dysfunction
in >60% of cross-validation experiments.

| # | GENE | FRACTION |
|---|---|---|
| 1 | NUSAP1 | 1 |
| 2 | CD24 | 1 |
| 3 | Propensity | 1 |
| 4 | CTSW | 1 |
| 5 | MPO | 1 |
| 6 | CFD | 1 |
| 7 | CXCL5 | 1 |
| 8 | NRGN | 1 |
| 9 | CKLF | 1 |
| 10 | GRAMD1C | 1 |
| 11 | GSEC | 1 |
| 12 | XCL1 | 1 |
| 13 | TAGAP | 1 |
| 14 | TOP1 | 0.95 |
| 15 | CST7 | 0.95 |
| 16 | CCDC92 | 0.95 |
| 17 | MTMR11 | 0.95 |
| 18 | TGFBI | 0.95 |
| 19 | CX3CR1 | 0.95 |
| 20 | SGPP1 | 0.95 |
| 21 | ASPM | 0.9 |
| 22 | PSAT1 | 0.9 |
| 23 | SLC26A8 | 0.9 |
| 24 | CEBPE | 0.9 |
| 25 | ELANE | 0.9 |
| 26 | DDX11L2 | 0.9 |
| 27 | MTF1 | 0.9 |
| 28 | TRBV27 | 0.85 |
| 29 | IL1R2 | 0.85 |
| 30 | BEND2 | 0.85 |
| 31 | IFIT2 | 0.85 |
| 32 | CENPW | 0.85 |
| 33 | PARP8 | 0.85 |
| 34 | FABP5 | 0.85 |
| 35 | KLRF1 | 0.85 |
| 36 | ATP9A | 0.85 |
| 37 | BCL6 | 0.8 |
| 38 | ASAP2 | 0.8 |
| 39 | LGALS2 | 0.8 |
| 40 | ARL4A | 0.8 |
| 41 | COX6C | 0.8 |
| 42 | LYSMD2 | 0.8 |
| 43 | TSPOAP1-AS1 | 0.8 |
| 44 | MAP3K7CL | 0.8 |
| 45 | F13A1 | 0.8 |
| 46 | ATP5PF | 0.8 |
| 47 | PCOLCE2 | 0.8 |
| 48 | ELOVL7 | 0.8 |
| 49 | PRC1 | 0.8 |
| 50 | TTK | 0.8 |
| 51 | LOC441081 | 0.8 |
| 52 | LMNB1 | 0.8 |
| 53 | CAPG | 0.8 |
| 54 | KLRD1 | 0.75 |
| 55 | IL10RB-DT | 0.75 |
| 56 | ASAP1-IT1 | 0.75 |
| 57 | CEACAM6 | 0.75 |
| 58 | PTTG1 | 0.75 |
| 59 | SH2D1B | 0.75 |
| 60 | TOP2A | 0.75 |
| 61 | DACH1 | 0.75 |
| 62 | IFI44L | 0.75 |
| 63 | CCL5 | 0.75 |
| 64 | ANLN | 0.75 |
| 65 | TBC1D7 | 0.75 |
| 66 | IQGAP1 | 0.75 |
| 67 | SLIRP | 0.75 |
| 68 | GNLY | 0.75 |
| 69 | CD177 | 0.75 |
| 70 | KIF11 | 0.75 |
| 71 | CLIC3 | 0.75 |
| 72 | CD1C | 0.7 |
| 73 | PRKAR2B | 0.7 |
| 74 | HCAR3 | 0.7 |
| 75 | IFI44 | 0.7 |
| 76 | IFIT1 | 0.7 |
| 77 | CDKN3 | 0.7 |
| 78 | CDC20 | 0.7 |
| 79 | RNASE3 | 0.7 |
| 80 | PLBD1 | 0.65 |
| 81 | ANKRD46 | 0.65 |
| 82 | TUBB1 | 0.65 |
| 83 | FCRL3 | 0.65 |
| 84 | PIK3CB | 0.65 |
| 85 | FPR2 | 0.65 |
| 86 | S100A12 | 0.65 |
| 87 | BLOC1S1 | 0.65 |
| 88 | MCEMP1 | 0.65 |
| 89 | EOMES | 0.65 |
| 90 | MAL | 0.65 |
| 91 | GBAP1 | 0.65 |
| 92 | HLA-DPA1 | 0.65 |
| 93 | ARG1 | 0.65 |
| 94 | SLA | 0.65 |
| 95 | PDZK1IP1 | 0.65 |
| 96 | CERT1 | 0.65 |
| 97 | RNASE2 | 0.65 |
| 98 | KIF14 | 0.6 |
| 99 | C12orf75 | 0.6 |
| 100 | ZDHHC19 | 0.6 |
| 101 | NCF4 | 0.6 |
| 102 | LY96 | 0.6 |
| 103 | C1QB | 0.6 |
| 104 | LGALS1 | 0.6 |
| 105 | BCL2A1 | 0.6 |
| 106 | FAM118B | 0.6 |
| 107 | HLA-DMB | 0.6 |
| 108 | PTX3 | 0.6 |
| 109 | AATBC | 0.6 |
| 110 | MANSC1 | 0.6 |
| 111 | AZU1 | 0.6 |
| 112 | SLC46A2 | 0.6 |
| 113 | CSTA | 0.6 |
| 114 | LCN2 | 0.6 |
| 115 | RGL4 | 0.6 |
| 116 | SEMA4A | 0.6 |
| 117 | MS4A3 | 0.6 |
| 118 | EMC2 | 0.6 |

TABLE 16
Top features correlated with day 7 CVS dysfunction
in > 60% of cross-validation experiments.

| # | GENE | FRACTION |
|---|---|---|
| 1 | ENO1 | 1 |
| 2 | CEACAM8 | 1 |
| 3 | GGH | 1 |
| 4 | SLC18B1 | 0.933 |
| 5 | GPBAR1 | 0.933 |
| 6 | CDKN3 | 0.933 |
| 7 | ABCA13 | 0.933 |
| 8 | NELL2 | 0.933 |
| 9 | MYBL1 | 0.933 |
| 10 | CD24 | 0.933 |
| 11 | KIF14 | 0.933 |
| 12 | LRG1 | 0.867 |
| 13 | PRG2 | 0.867 |
| 14 | ZSCAN26 | 0.867 |
| 15 | ST6GALNAC3 | 0.867 |
| 16 | ANLN | 0.867 |
| 17 | Propensity | 0.8 |
| 18 | HMGB3 | 0.8 |
| 19 | LDHA | 0.733 |
| 20 | PRR11 | 0.733 |
| 21 | ELANE | 0.733 |
| 22 | HCG26 | 0.733 |
| 23 | SIGLEC17P | 0.733 |
| 24 | FCER1A | 0.733 |
| 25 | DPEP2 | 0.667 |
| 26 | SGK1 | 0.667 |
| 27 | NUCB2 | 0.667 |
| 28 | CHRM3-AS2 | 0.667 |
| 29 | PRTN3 | 0.667 |
| 30 | GPRASP1 | 0.667 |
| 31 | RNASE3 | 0.667 |
| 32 | HLA-DMA | 0.667 |
| 33 | CKAP2L | 0.667 |
| 34 | HCP5 | 0.667 |
| 35 | SLC39A13 | 0.667 |
| 36 | AMIGO1 | 0.6 |
| 37 | GBE1 | 0.6 |
| 38 | MAFG | 0.6 |
| 39 | IFI44L | 0.6 |
| 40 | NUF2 | 0.6 |
| 40 | SHCBP1 | 0.6 |
| 41 | CTSL | 0.6 |
| 42 | ASPM | 0.6 |
| 43 | TGFBI | 0.6 |
| 44 | HLA-DPA1 | 0.6 |
| 45 | CHIT1 | 0.6 |
| 46 | LYSMD2 | 0.6 |
| 47 | SFXN1 | 0.6 |
| 48 | STIL | 0.6 |
| 49 | FANCI | 0.6 |
| 50 | PLA2G4A | 0.6 |

TABLE 17
Top features correlated with day 7 respiratory dysfunction
in >60% of cross-validation experiments.

| # | GENE | FRACTION |
|---|---|---|
| 1 | NARF | 1 |
| 2 | CENPW | 1 |
| 3 | SAMSN1 | 1 |
| 4 | PRTN3 | 1 |
| 5 | CD24 | 1 |
| 6 | CLEC4D | 1 |
| 7 | RETN | 1 |
| 8 | SERPINB2 | 1 |
| 9 | DDX11L2 | 1 |
| 10 | VNN1 | 1 |
| 11 | Propensity | 0.933 |
| 12 | PDZK1IP1 | 0.933 |
| 13 | LDHA | 0.933 |
| 14 | LCN2 | 0.933 |
| 15 | CCL5 | 0.933 |
| 16 | FCER1A | 0.933 |
| 17 | TRBV27 | 0.933 |
| 18 | ECRP | 0.933 |
| 19 | DUSP13 | 0.867 |
| 20 | ARG1 | 0.867 |
| 21 | NCAPG | 0.867 |
| 22 | SMPDL3A | 0.867 |
| 23 | KIF20A | 0.867 |
| 24 | CPA3 | 0.867 |
| 25 | ALOX5AP | 0.867 |
| 26 | ATP2B1-AS1 | 0.867 |
| 27 | DHCR7 | 0.867 |
| 28 | CHIT1 | 0.867 |
| 29 | HSPB1 | 0.867 |
| 30 | C1QB | 0.867 |
| 31 | HCAR3 | 0.867 |
| 32 | SLFN5 | 0.867 |
| 33 | PADI4 | 0.867 |
| 34 | XCL1 | 0.867 |
| 35 | MCEMP1 | 0.867 |
| 36 | EXOSC4 | 0.867 |
| 37 | LTF | 0.867 |
| 38 | NUSAP1 | 0.867 |
| 39 | CTSW | 0.867 |
| 40 | PFKFB2 | 0.867 |
| 41 | GADD45A | 0.867 |
| 42 | PGLYRP1 | 0.867 |
| 43 | FGF13 | 0.8 |
| 44 | GPI | 0.8 |
| 45 | MTARC1 | 0.8 |
| 46 | CYSTM1 | 0.8 |
| 47 | ADGRE3 | 0.8 |
| 48 | UHRF1 | 0.8 |
| 49 | GPR160 | 0.8 |
| 50 | IFIT2 | 0.8 |
| 51 | GPR84 | 0.8 |
| 52 | OLAH | 0.8 |
| 53 | LRIG3 | 0.8 |
| 54 | ARL4A | 0.8 |
| 55 | CACNA2D3 | 0.8 |
| 56 | KIF4A | 0.8 |
| 57 | GJB6 | 0.8 |
| 58 | HGF | 0.8 |
| 59 | PROK2 | 0.8 |
| 60 | BST1 | 0.8 |
| 61 | FOLR3 | 0.8 |
| 62 | TRAT1 | 0.8 |
| 63 | MPO | 0.733 |
| 64 | MMP8 | 0.733 |
| 65 | SLC25A40 | 0.733 |
| 66 | UPP1 | 0.733 |
| 67 | GYG1 | 0.733 |
| 68 | SGK1 | 0.733 |
| 69 | CDC20 | 0.733 |
| 70 | FGL2 | 0.733 |
| 71 | CD160 | 0.733 |
| 72 | PLAC8 | 0.733 |
| 73 | GCA | 0.733 |
| 74 | CLEC5A | 0.733 |
| 75 | LY9 | 0.733 |
| 76 | PNPLA6 | 0.733 |
| 77 | SULF2 | 0.733 |
| 78 | SGPP1 | 0.733 |
| 79 | SYTL2 | 0.733 |
| 80 | NKG7 | 0.733 |
| 79 | ANKRD37 | 0.733 |
| 80 | S100A12 | 0.733 |
| 81 | HLA-DMB | 0.733 |
| 82 | CKS2 | 0.733 |
| 83 | ITM2A | 0.733 |
| 84 | GNA15 | 0.733 |
| 85 | SLC22A4 | 0.733 |
| 86 | CEBPE | 0.733 |
| 87 | CCR3 | 0.733 |
| 88 | MME | 0.733 |
| 89 | PFKFB3 | 0.733 |
| 90 | CD200 | 0.733 |
| 91 | NRN1 | 0.733 |
| 92 | FCMR | 0.733 |
| 93 | CPEB4 | 0.733 |
| 94 | KL | 0.733 |
| 95 | FCAR | 0.733 |
| 96 | RTN1 | 0.733 |
| 97 | LAMP3 | 0.667 |
| 98 | MAML2 | 0.667 |
| 99 | LGALS1 | 0.667 |
| 100 | KLRC3 | 0.667 |
| 101 | ELANE | 0.667 |
| 102 | ADAMTS3 | 0.667 |
| 103 | KCNE1 | 0.667 |
| 104 | MEF2C | 0.667 |
| 105 | GZMK | 0.667 |
| 106 | ADA2 | 0.6 |
| 107 | STOM | 0.6 |
| 108 | MAFG | 0.6 |
| 109 | CCNA1 | 0.6 |
| 110 | HIP1 | 0.6 |
| 111 | RNF182 | 0.6 |
| 112 | IFIT1 | 0.6 |

TABLE 18
Top features correlated with day 7 renal dysfunction.

| # | GENE | FRACTION |
|---|---|---|
| 1 | CTSW | 1 |
| 2 | NRGN | 1 |
| 3 | Propensity | 1 |
| 4 | TAGAP | 0.95 |
| 5 | NUSAP1 | 0.95 |
| 6 | PSAT1 | 0.9 |
| 7 | ELANE | 0.9 |
| 8 | CTSG | 0.85 |
| 9 | CEACAM6 | 0.85 |
| 10 | DACH1 | 0.85 |
| 11 | E2F8 | 0.85 |
| 12 | CYSTM1 | 0.85 |
| 13 | MPO | 0.85 |
| 14 | ATP9A | 0.85 |
| 15 | CA4 | 0.8 |
| 16 | PCOLCE2 | 0.8 |
| 17 | CENPW | 0.8 |
| 18 | CFD | 0.8 |
| 19 | GNG11 | 0.8 |
| 20 | PHGDH | 0.8 |
| 21 | F13A1 | 0.8 |
| 22 | NCAPG | 0.8 |
| 23 | MELK | 0.8 |
| 24 | BEX1 | 0.8 |
| 25 | GPBAR1 | 0.75 |
| 26 | CDK1 | 0.75 |
| 27 | GSEC | 0.75 |
| 28 | GGH | 0.75 |
| 29 | IFIT1 | 0.75 |
| 30 | CD24 | 0.75 |
| 31 | KLRD1 | 0.75 |
| 32 | ADAMTS3 | 0.75 |
| 33 | S100P | 0.75 |
| 34 | MTMR11 | 0.75 |
| 35 | PADI4 | 0.75 |
| 36 | ELOVL7 | 0.75 |
| 37 | LINC01003 | 0.75 |
| 38 | RETN | 0.75 |
| 39 | KIF20A | 0.7 |
| 41 | MKI67 | 0.7 |
| 42 | IL10RB-DT | 0.7 |
| 43 | C1QA | 0.7 |
| 44 | PRKAR2B | 0.7 |
| 45 | PRC1 | 0.7 |
| 46 | IL1R2 | 0.7 |
| 47 | TCN1 | 0.7 |
| 48 | DDX11L2 | 0.7 |
| 49 | LYSMD2 | 0.7 |
| 50 | CAPG | 0.7 |
| 51 | ARG1 | 0.65 |
| 52 | TGFBI | 0.65 |
| 53 | CR1 | 0.65 |
| 54 | RGL4 | 0.65 |
| 55 | HP | 0.65 |
| 56 | HK3 | 0.65 |
| 57 | PIK3CB | 0.65 |
| 58 | PRMT6 | 0.65 |
| 59 | JUN | 0.65 |
| 60 | NFE2 | 0.65 |
| 61 | RTN1 | 0.6 |
| 62 | PRG2 | 0.6 |
| 63 | LCN2 | 0.6 |
| 64 | ARL4A | 0.6 |
| 65 | TRBV27 | 0.6 |
| 66 | ASPM | 0.6 |
| 67 | RFLNB | 0.6 |
| 68 | MS4A1 | 0.6 |
| 69 | CST7 | 0.6 |
| 70 | ANLN | 0.6 |
| 71 | PLBD1 | 0.6 |
| 72 | SGPP1 | 0.6 |
| 73 | IQGAP1 | 0.6 |
| 74 | IFIT2 | 0.6 |
| 75 | TUBB1 | 0.6 |

TABLE 19
Differentially expressed genes among day 3 cardiovascular
dysfunction not shared by the MODS signature.

| DEGs unique to patients with day 3 CVS dysfunction | | | Top features | Fraction |
|---|---|---|---|---|
| ADAM9 | HBZ | PROK2 | CCR1 | 0.971 |
| AGTRAP | HCK | PRSS33 | GPR34 | 0.971 |
| AIM2 | HLX | QPCT | PDSS1 | 0.914 |
| ALAS1 | IER3 | RAB24 | LILRA3 | 0.886 |
| ANXA1 | IFI35 | ROPN1L | PIM3 | 0.857 |
| AQP9 | IGF2R | SDHAF3 | LINC00597 | 0.771 |
| ARL8A | IGSF6 | SELENBP1 | LINC01000 | 0.714 |
| ASAP1-IT1 | IL10RB | SH3GLB1 | MAP3K20 | 0.714 |
| ASCC2 | IL7R | SHOC1 | PROK2 | 0.714 |
| ATXN1 | KCNJ15 | SLA | MICOS10P1 | 0.686 |
| BATF | LILRA3 | SLC22A4 | SHOC1 | 0.657 |
| C5AR1 | LILRB3 | SLC25A39 | ASAP1-IT1 | 0.629 |
| CARD8-AS1 | LIMK2 | SNCA | SOCS3 | 0.629 |
| CCR1 | LINC00266-1 | SOCS3 | | |
| CD14 | LINC00597 | STRADB | | |
| CD300LF | LINC01000 | TCTEX1D1 | | |
| CD59 | LINC01093 | TIPARP | | |
| CDC42EP3 | LINC01127 | TLR2 | | |
| CHSY1 | LMNB1 | TLR8 | | |
| CKLF | MAP3K20 | TRIM58 | | |
| CPD | MARCKS | TRPS1 | | |
| CREB5 | MCTP1 | TSHZ3 | | |
| CXCL1 | MICOS10P1 | TXK | | |
| DHRS13 | MIR3945HG | VASP | | |
| DIAPH2 | MLKL | VMP1 | | |
| DMTN | MSRB1 | VPS9D1 | | |
| EPB42 | MTF1 | WSB1 | | |
| F5 | NABP1 | ZNF438 | | |
| FBXO6 | NCF4 | | | |
| FFAR2 | NFIL3 | | | |
| GK | NIBAN1 | | | |
| GK3P | ODC1 | | | |
| GLT1D1 | PADI2 | | | |
| GPR141 | PDSS1 | | | |
| GPR146 | PIK3AP1 | | | |
| GPR34 | PIK3CB | | | |
| GSTO1 | PIM3 | | | |
| H2BC9 | PLA2G4A | | | |
| H2BS1 | PPP1R3D | | | |
| H4C8 | PRKCD | | | |

TABLE 20
Differentially expressed genes among day 3 respiratory
dysfunction not shared by the MODS signature.

| DEGs unique to patients with day 3 respiratory dysfunction | | | | | Top features | Fraction |
|---|---|---|---|---|---|---|
| ACSL4 | CTSA | IER3 | NABP1 | S100A11 | HSPB1 | 0.857 |
| ADAM9 | DGAT2 | IFNGR1 | NAMPT | SDCBPP2 | LILRA3 | 0.857 |
| AGTPBP1 | DHRS13 | IGSF6 | NCF4 | SDHAF3 | GSTO1 | 0.829 |
| AGTRAP | DNASE1L1 | IL10RB | NFE4 | SELENBP1 | H2BS1 | 0.743 |
| AIM2 | DOK3 | IL4R | NFIL3 | SH3GLB1 | RNF182 | 0.743 |
| ALDOA | EDEM2 | IL7R | NIBAN1 | SHKBP1 | UBE2F | 0.743 |
| ALYREF | EIF4E3 | IMPA2 | NLRP12 | SIGLEC10 | POMP | 0.714 |
| ANXA1 | EPB42 | IRAG1 | NME8 | SIRPA | PDZK1IP1 | 0.686 |
| ANXA5 | ETS2 | JAK2 | NOP10 | SIRPB2 | H2BC9 | 0.657 |
| APOBR | EXOC6 | JUNB | NSUN7 | SLA | NOP10 | 0.629 |
| AQP9 | F5 | KIAA0930 | OAT | SLC22A15 | | |
| ARL8A | FAM126B | LACTB | OSTF1 | SLC22A4 | | |
| ARPC1B | FAM160A2 | LAIR1 | OTULINL | SLC25A39 | | |
| ATP6V0D1 | FAR1 | LAMTOR5 | P2RX1 | SLC25A40 | | |
| BATF | FBXO6 | LILRA3 | PADI2 | SNX3 | | |
| C1GALT1C1 | FERMT3 | LILRA6 | PDGFC | SOCS3 | | |
| C1orf162 | FES | LILRB1 | PDZD8 | STRADB | | |
| C5AR1 | FFAR2 | LILRB2 | PDZK1IP1 | TBXAS1 | | |
| CAMP | FPR1 | LILRB3 | PECR | TCTEX1D1 | | |
| CARD16 | GATA3 | LIMK2 | PGM2 | TIMP2 | | |
| CCR1 | GK | LINC00266-1 | PIK3AP1 | TIPARP | | |
| CD14 | GK3P | LINC01000 | PIK3CB | TLR2 | | |
| CD1C | GLIPR2 | LINC01093 | PIM3 | TLR4 | | |
| CD27 | GLRX | LINC01127 | PLA2G4A | TLR8 | | |
| CD300LF | GLT1D1 | LMNB1 | POMP | TMEM260 | | |
| CD58 | GMFG | LPCAT2 | PPM1M | TPST2 | | |
| CD59 | GNAQ | MAN1A1 | PPP1R3D | TSHZ3 | | |
| CD82 | GNB2 | MAP3K20 | PRAM1 | TXK | | |
| CDC42EP3 | GNG5 | MARCKS | PRKCD | UBE2F | | |
| CDK5RAP2 | GNS | MCTP1 | PROK2 | VASP | | |
| CEBPA | GSTO1 | MEF2A | PRSS33 | VIM | | |
| CEBPB | H2BC6 | METRNL | QPCT | VNN2 | | |
| CERT1 | H2BC9 | MGST1 | RAB32 | VPS9D1 | | |
| CHMP2A | H2BS1 | MILR1 | RALB | VSIR | | |
| CHSY1 | H4C8 | MLKL | RARA-AS1 | WSB1 | | |
| CKLF | HCK | MMADHC | RHOG | ZNF438 | | |
| CPD | HLX | MSRB1 | RNF182 | | | |
| CSF2RA | HPSE | MSRB2 | ROPN1L | | | |
| CSF3R | HSPB1 | MTARC1 | RTN3 | | | |

TABLE 21
Differentially expressed genes among day 3 renal
dysfunction not shared by the MODS signature.

| DEGs unique to patients with day 3 renal dysfunction | | Gene | Fraction |
|---|---|---|---|
| ABHD2 | PRAM1 | CXCL5 | 1 |
| AIM2 | PRKAR2B | CKLF | 1 |
| ASAP1-IT1 | PSAT1 | NRGN | 1 |
| ASAP2 | SELENBP1 | CCDC92 | 0.95 |
| BEND2 | SH3GLB1 | TOP1 | 0.95 |
| C5AR1 | SHPRH | PSAT1 | 0.9 |
| CAVIN2 | SIGLEC10 | MTF1 | 0.9 |
| CCDC92 | SLA | DDX11L2 | 0.9 |
| CCR1 | SLC28A3 | BEND2 | 0.85 |
| CD14 | STRADB | ASAP2 | 0.8 |
| CD1C | TIMP2 | F13A1 | 0.8 |
| CD300LF | TOP1 | LMNB1 | 0.8 |
| CD69 | TSHZ3 | ELOVL7 | 0.8 |
| CEACAM4 | TUBB1 | IQGAP1 | 0.75 |
| CERT1 | UBE2E2 | ASAP1- | 0.75 |
| | | IT1 | |
| CKLF | ZNF438 | CD1C | 0.7 |
| CMPK2 | ZYX | PRKAR2B | 0.7 |
| CXCL5 | | CERT1 | 0.65 |
| DDX11L2 | | PIK3CB | 0.65 |
| ELOVL7 | | SLA | 0.65 |
| EPB42 | | TUBB1 | 0.65 |
| F13A1 | | PDZK1IP1 | 0.65 |
| FCRL1 | | NCF4 | 0.6 |
| GMFG | | | |
| GSTO1 | | | |
| HCK | | | |
| IQGAP1 | | | |
| LILRB3 | | | |
| LMNB1 | | | |
| MILR1 | | | |
| MTF1 | | | |
| NCF4 | | | |
| NFIL3 | | | |
| NIBAN1 | | | |
| NOC3L | | | |
| NRGN | | | |
| PDZK1IP1 | | | |
| PF4V1 | | | |
| PIK3CB | | | |

TABLE 22
Differentially expressed genes among day 7 cardiovascular
dysfunction not shared by the MODS signature.

| | | | | Top | |
|---|---|---|---|---|---|
| DEGs unique to patients with day 7 CVS dysfunction | | | | features | Fraction |
| ADAM9 | GINS1 | NCF4 | SNRPG | GPBAR1 | 0.933 |
| ADIPOR1 | GLRX2 | NDC80 | SPC25 | ZSCAN26 | 0.867 |
| AGTPBP1 | GMNN | NDUFA4 | STIL | HMGB3 | 0.8 |
| ALAS1 | GMPR | NEK2 | STRADB | PRR11 | 0.733 |
| ALDOA | GPBAR1 | NME1 | TBCA | HCG26 | 0.733 |
| ALYREF | GPR141 | NUCB2 | TCTEX1D1 | SIGLEC17P | 0.733 |
| AMIGO1 | GPR146 | OIP5 | TK1 | NUCB2 | 0.667 |
| ANXA1 | GSTO1 | P2RX1 | TMEM52B | | |
| ARL8A | GYS1 | P4HB | TPRKB | | |
| ASAP1-IT1 | HAT1 | PAM | TRIM58 | | |
| ASCC2 | HCG26 | PCNA | TRMT6 | | |
| AURKA | HCK | PDZD8 | TUBA4A | | |
| CBX7 | HMGB3 | PDZK1IP1 | TUBB2A | | |
| CCDC125 | HOXB2 | PGM1 | TUBG1 | | |
| CCL4 | HTATSF1P2 | PIK3CB | TYMS | | |
| CCNA2 | IER3 | PIWIL4 | USP9Y | | |
| CCNE2 | INHBA | PLA2G4A | VAT1 | | |
| CD59 | KBTBD6 | POLE2 | WSB1 | | |
| CDCA5 | KIF15 | PRAM1 | ZSCAN26 | | |
| CDK1 | KIF2C | PRDX6- | ZUP1 | | |
| | | AS1 | | | |
| CENPE | KPNA2 | PROS1 | ZWILCH | | |
| CHCHD7 | LILRA3 | PRR11 | | | |
| CKLF | LINC00597 | PRUNE2 | | | |
| CKS1B | LNPK | PSAT1 | | | |
| CPD | LOC102724587 | PSMA4 | | | |
| CPNE3 | LY86 | PSMD14 | | | |
| CSGALNACT1 | MAP3K14 | PTGER2 | | | |
| CTSA | MARCKS | RACGAP1 | | | |
| DEPDC1 | MBNL3 | RAD51AP1 | | | |
| DIAPH2 | MICOS10P1 | RNF182 | | | |
| DMTN | MICU3 | RPS4Y1 | | | |
| ELOC | MIR646HG | SDHAF3 | | | |
| EPB42 | MMADHC | SEC24A | | | |
| EPHX2 | MMP27 | SEC61G | | | |
| ERO1A | MRPL13 | SELENBP1 | | | |
| ETFDH | MRPL22 | SESN2 | | | |
| FANCI | MTARC1 | SGO2 | | | |
| FBX06 | NAA38 | SIGLEC17P | | | |
| FOXM1 | NCAPG2 | SLC25A39 | | | |

TABLE 23
Differentially expressed genes among day 7 respiratory
dysfunction not shared by the MODS signature.

| DEGs unique to patients with day 7 respiratory dysfunction | | Top features | Fraction |
|---|---|---|---|
| ADAM9 | NME8 | DDX11L2 | 1 |
| AGTPBP1 | P2RX1 | PDZK1IP1 | 0.933 |
| AGTRAP | PDZD8 | HSPB1 | 0.867 |
| ANXA1 | PDZK1IP1 | MTARC1 | 0.8 |
| ASGR2 | PLA2G4A | PROK2 | 0.8 |
| Clorf162 | PRAM1 | SLC25A40 | 0.733 |
| CCR1 | PROK2 | SLC22A4 | 0.733 |
| CD14 | RNF182 | RNF182 | 0.667 |
| CD27 | RTN3 | ADAM9 | 0.667 |
| CD59 | SH3GLB1 | TXK | 0.667 |
| CD82 | SIGLEC10 | ASGR2 | 0.667 |
| CKLF | SLC22A4 | | |
| CTSA | SLC25A40 | | |
| DDX11L2 | SNX3 | | |
| EIF1AY | SOCS3 | | |
| FES | TIMP2 | | |
| GK3P | TLR8 | | |
| GLT1D1 | TOP1 | | |
| H2BC9 | TXK | | |
| HCK | VPS9D1 | | |
| HLX | | | |
| HSPB1 | | | |
| IGSF6 | | | |
| IL7R | | | |
| IMPA2 | | | |
| KIAA0930 | | | |
| LILRA3 | | | |
| LILRB2 | | | |
| LILRB3 | | | |
| LIMK2 | | | |
| LINC01127 | | | |
| LMNB1 | | | |
| MILR1 | | | |
| MTARC1 | | | |
| MTF1 | | | |
| NCF4 | | | |
| NFIL3 | | | |
| NIBAN1 | | | |
| NLRP12 | | | |

TABLE 24
Differentially expressed genes among day 7 renal
dysfunction not shared by the MODS signature.

| DEGs unique to patients with day 7 renal dysfunction | | Top features | Fraction |
|---|---|---|---|
| AIM2 | LILRB3 | PSAT1 | 1 |
| AMIGO1 | LINC01003 | NRGN | 1 |
| ASAP2 | LMNB1 | GPBAR1 | 0.8 |
| BEND2 | MRPL13 | CDK1 | 0.8 |
| C5AR1 | MTF1 | F13A1 | 0.8 |
| CAMP | NCF4 | GNG11 | 0.8 |
| CAVIN2 | NDUFAF1 | LINC01003 | 0.75 |
| CD14 | NFE4 | ELOVL7 | 0.75 |
| CDK1 | NFIL3 | DDX11L2 | 0.7 |
| CERT1 | NIBAN1 | PRKAR2B | 0.7 |
| CKLF | NME1 | | |
| CMPK2 | NRGN | | |
| CPD | PDZK1IP1 | | |
| CXCL10 | PECR | | |
| CXCL5 | PF4V1 | | |
| DBI | PIK3CB | | |
| DDX11L2 | PRKAR2B | | |
| ELOVL7 | PROK2 | | |
| EPB42 | PSAT1 | | |
| ERO1A | RACGAP1 | | |
| F13A1 | RPS4Y1 | | |
| F5 | SELENBP1 | | |
| FHIT | SGO2 | | |
| GINS1 | SH3GLB1 | | |
| GMFG | SIGLEC10 | | |
| GMNN | SLA | | |
| GNG11 | SLC25A39 | | |
| GPBAR1 | SMIM5 | | |
| GPR146 | SNCA | | |
| GSTO1 | SNRPG | | |
| HCK | SPC25 | | |
| HSPE1 | STRADB | | |
| IGF2R | TIMP2 | | |
| IGHD | TRIM58 | | |
| IGK | TSHZ3 | | |
| IMPA2 | TUBB1 | | |
| IQGAP1 | XIST | | |
| LDLRAP1 | ZNF438 | | |
| LILRA3 | ZYX | | |

### Example 9

### Identification of Novel MODS Endotypes with Distinct Clinical Features

The top 50 features correlated with persistent MODS were used to derive new pediatric sepsis subclasses. These features are: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, and MS4A4A.

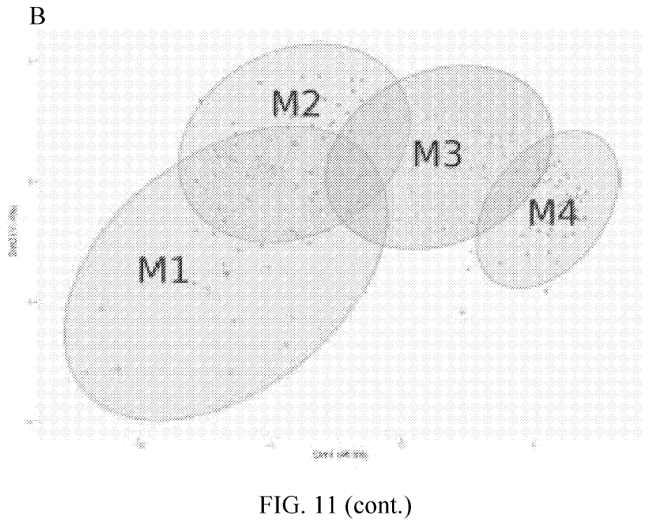

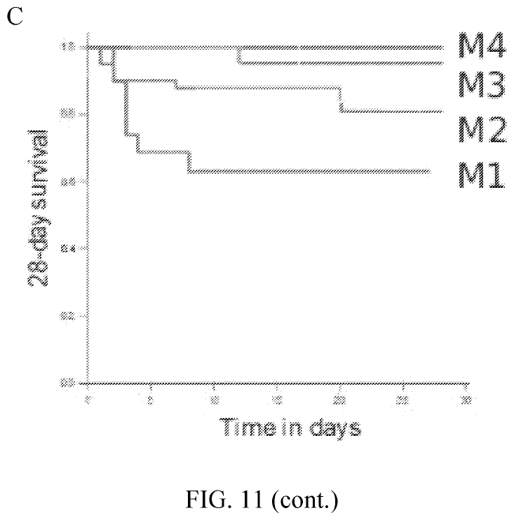

FIG. 11 summarizes the results. Four groups were identified: the M1 (n=23) and M2 subclasses (n=63) had high rates of MODS, at 74% and 41% respectively, in comparison with 2% in the M3 subclass and 0% in the M4 subclass, as shown in Table 25. All healthy controls, patients with SIRS, and patients with sepsis without organ dysfunction were clustered in the M4 subclass. M1 and M2 subclasses had significantly lower survival relative to M3 and M4 subclasses (p<0.01).

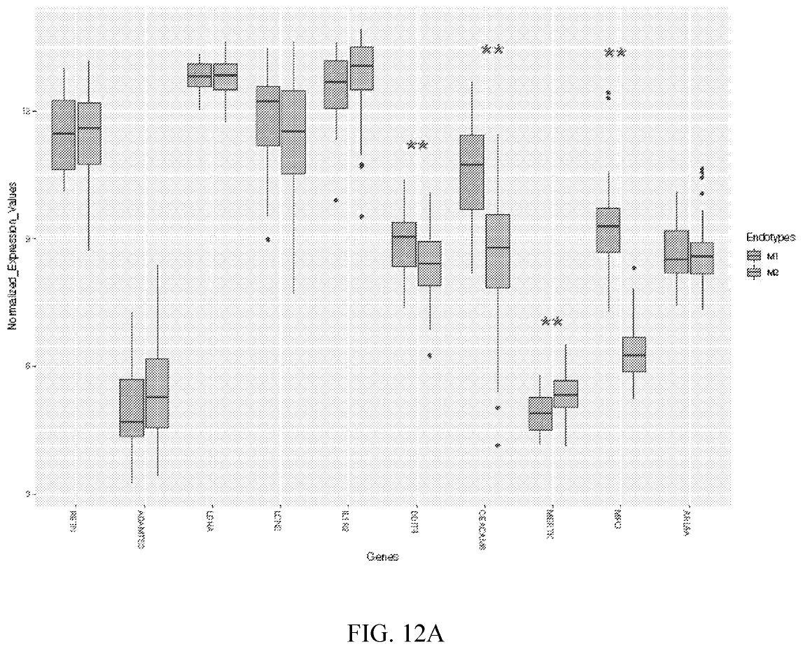

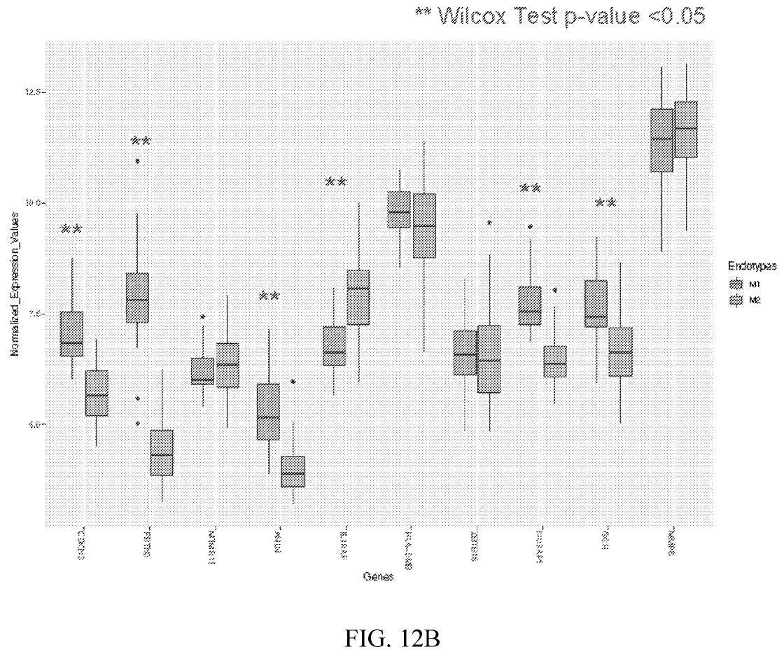

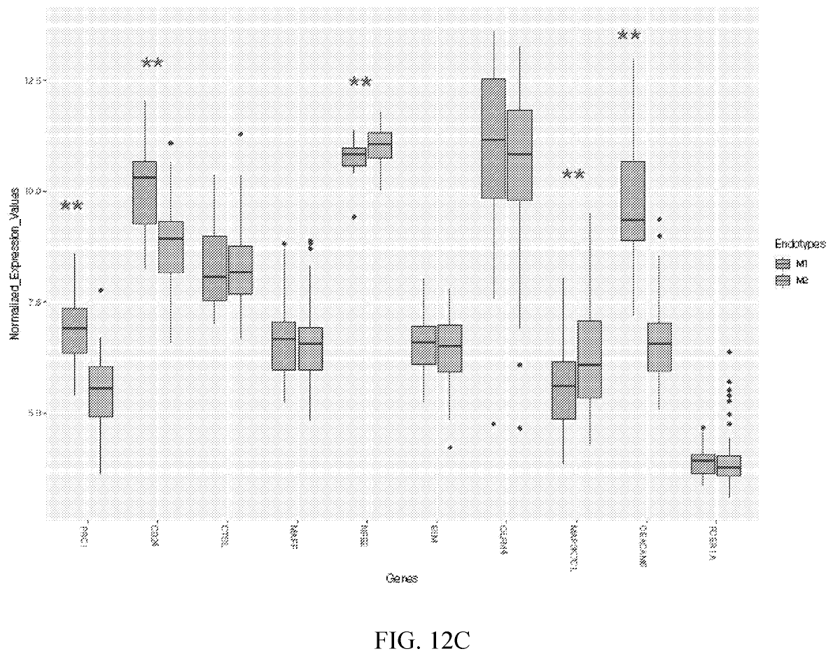

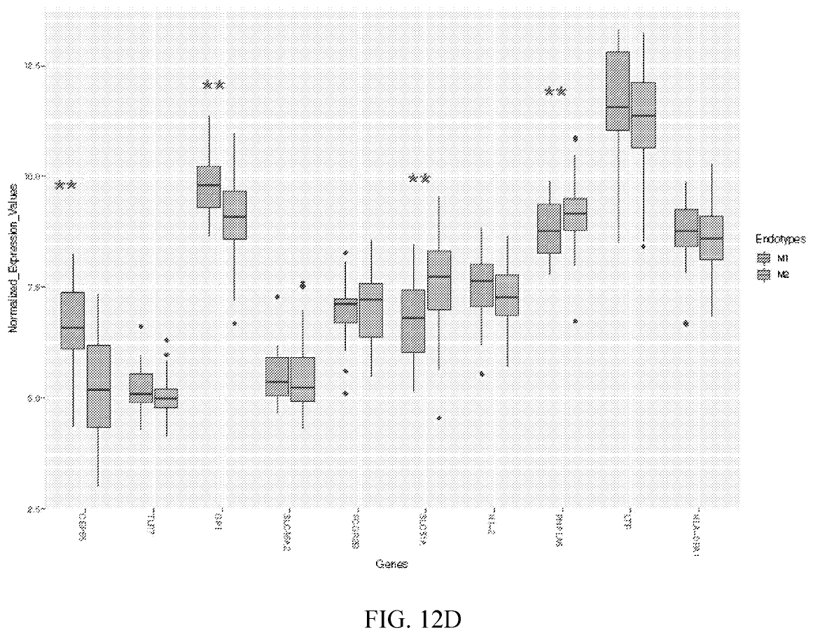

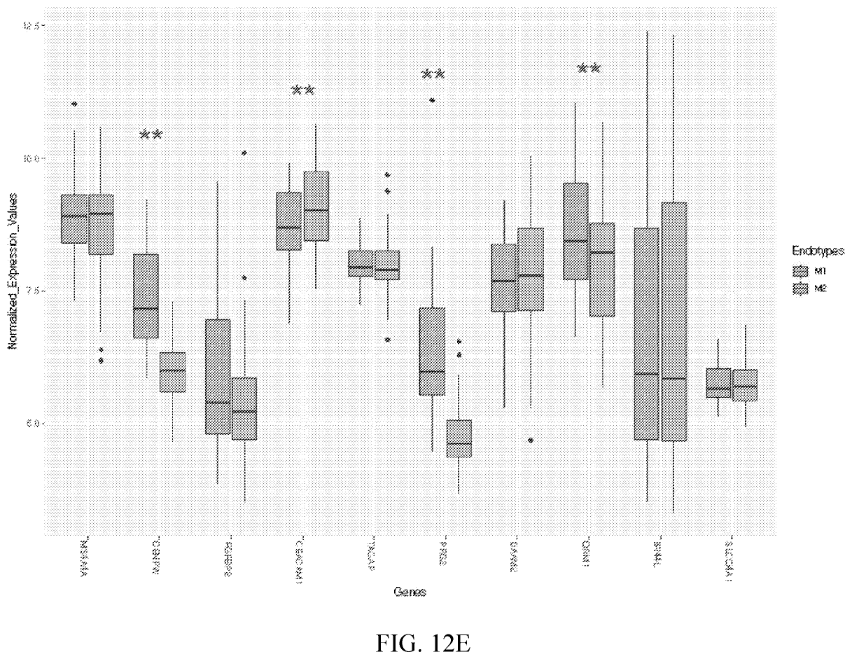

A comparison of the two newly derived MODS endotypes M1 and M2 is shown Table 26. Patients belonging to the M1 endotype were younger relative to the M2 endotype with no other meaningful differences in demographic data between groups. Relative to patients with membership in the M2 endotype, those with M1 endotype were less likely to be prescribed adjunctive steroids by the treating team and had significantly higher organ support requirements, including vasoactive use, mechanical ventilation, and renal replacement therapy. As detailed in Table 27, use of adjunctive steroids was not independently associated with persistent MODS or 28-day mortality in either of the M1 or M2 endotypes. The p value for the interaction term between endotype X receipt of steroids in the M1 relative to the M2 endotype was 0.56 for 28-day mortality and 0.97 for day 7 multiple organ dysfunction. Reactome analyses of these 50 features used to derive sepsis subclasses are detailed in Table 28. Beyond alterations in the innate and adaptive immune systems, the roles of which are well established in sepsis, a major role of transcription factor RUNX1 was identified. Differential expression of the 50 genes used to determine patient subclasses between M1 and M2 endotypes are shown in FIG. 12. Microarray data are normalized using Robust Multiarray/Multichip Average (RMA) normalization, which is a technique understood and appreciated by those skilled in the art. For further detail, see Bolstad, B. M., Irizarry R. A., Astrand M., and Speed, T. P. (2003), A Comparison of Normalization Methods for High Density Oligonucleotide Array Data Based on Bias and Variance. Bioinformatics 19(2):185-193; Rafael. A. Irizarry, Benjamin M. Bolstad, Francois Collin, Leslie M. Cope, Bridget Hobbs and Terence P. Speed (2003), Summaries of Affymetrix GeneChip probe level data. Nucleic Acids Research 31(4):e15; Irizarry, RA, Hobbs, B, Collin, F, Beazer-Barclay, YD, Antonellis, KJ, Scherf, U, Speed, TP (2003) Exploration, Normalization, and Summaries of High Density Oligonucleotide Array Probe Level Data. Biostatistics, Vol. 4, Number 2: 249-264; these references are incorporated by reference herein in their entirety. A summary is also provided at https<colon slash slash>bmbolstad<dot>com<slash>misc<slash>ComputeRMAFAQ<slash>ComputeRMAFAQ<dot>html.

TABLE 25
Comparison of demographic and outcome variables by newly derived sepsis subclasses.

| | M1 | | M2 | | M3 | | M4 | | P value |
|---|---|---|---|---|---|---|---|---|---|
| N = 201 | 23 | | 63 | | 53 | | 62 | | |
| Age (years) | 1.4 | (0.1, 4.5) | 2.4 | (1.1, 6.5) | 2.9 | (1.3, 6.0) | 2.9 | (1.2, 5.3) | 0.09 |
| Sex, male | 14 | (60.9%) | 38 | (60.3%) | 27 | (50.9%) | 32 | (51.6%) | 0.64 |
| PRISM-III | 22 | (12, 26) | 14 | (10, 20) | 11 | (7, 16) | 0 | (0, 0) | <0.01 |
| Day 1 VIS | 5 | (0, 50) | 10 | (0, 33) | 3 | (0,10) | 0 | (0, 11) | 0.02 |
| 28-day mortality | 9 | (39.1%) | 8 | (12.7%) | 1 | (1.8%) | 0 | (0%) | <0.01 |
| Day 7 MODS | 17 | (73.9%) | 26 | (41.3%) | 1 | (1.9%) | 0 | (0%) | <0.01 |
| PICU free days | 17 | (1, 22) | 19 | (12, 23) | 23 | (17, 25) | 24 | (18, 26) | 0.01 |
| PICU LOS | 8 | (2, 12) | 7 | (4, 12) | 5 | (3, 11) | 4 | (2, 9) | 0.32 |
| Hospital LOS | 10 | (3, 25) | 14 | (7, 32) | 11 | (8, 18) | 9 | (6, 20) | 0.29 |
| Steroid use | 4 | (17.4%) | 27 | (42.8%) | 10 | (18.9%) | 1 | (1.6%) | <0.01 |
| D3 vasoactive use | 20 | (90.9%) | 38 | (64.4%) | 14 | (35.9%) | 6 | (21.4%) | <0.01 |
| D7 vasoactive use | 16 | (72.7%) | 15 | (28.3%) | 3 | (9.1%) | 0 | (0%) | <0.01 |
| Day 3 MV | 19 | (86.3%) | 38 | (66.6%) | 17 | (36.9%) | 4 | (6.9%) | <0.01 |
| Day 7 MV | 18 | (81.8%) | 28 | (49.1%) | 4 | (9.7%) | 1 | (1.7%) | <0.01 |
| Day 3 RRT | 11 | (47.8%) | 10 | (17.5%) | 1 | (2.2%) | 0 | (0%) | <0.01 |
| Day 7 RRT | 12 | (54.5%) | 10 | (17.5%) | 1 | (2.2%) | 0 | (0%) | <0.01 |

LOS: Length of stay; MV: Mechanical ventilation; RRT: renal replacement therapy

TABLE 26
Comparison of demographic and outcome variables
by newly derived MODS endotypes.

| | | |
|---|---|---|
| M1 | M2 | P value |

| | |
|---|---|
| 23 | 63 |

| | | | | | |
|---|---|---|---|---|---|
| Age (years) | 1.4 | (0.1, 4.5) | 2.4 | (1.1, 6.5) | 0.04 |
| Sex, male | 14 | (60.9%) | 38 | (60.3%) | 0.87 |
| PRISM-III | 22 | (12, 26) | 14 | (10, 20) | 0.06 |
| Day 1 VIS | 5 | (0, 50) | 10 | (0, 33) | 0.73 |
| 28-day mortality | 9 | (39.1%) | 8 | (12.7%) | 0.01 |
| Day 7 MODS | 17 | (73.9%) | 26 | (41.3%) | |
| PICU free days | 17 | (1, 22) | 19 | (12, 23) | 0.3 |
| PICU LOS | 8 | (2, 12) | 7 | (4, 12) | 0.52 |
| Hospital LOS | 10 | (3, 25) | 14 | (7, 32) | 0.62 |
| Steroid use | 4 | (17.4%) | 27 | (42.8%) | 0.03 |
| D3 vasoactive use | 20 | (90.9%) | 38 | (64.4%) | 0.02 |
| D7 vasoactive use | 16 | (72.7%) | 15 | (28.3%) | <0.01 |
| Day 3 MV | 19 | (86.3%) | 38 | (66.6%) | 0.17 |
| Day 7 MV | 18 | (81.8%) | 28 | (49.1%) | 0.02 |
| Day 3 RRT | 11 | (47.8%) | 10 | (17.5%) | <0.01 |
| Day 7 RRT | 12 | (54.5%) | 10 | (17.5%) | <0.01 |

LOS: Length of stay; MV: Mechanical ventilation; RRT: renal replacement therapy

TABLE 27
Logistic regression to test the association between receipt of adjuvant
corticosteroids and clinical outcomes by MODS endotypes.

| Term | Coeff. | SE Coeff. | P -value |
|---|---|---|---|
| 28-day mortality | | | |
| Constant | −1.83 | 0.48 | 0 |
| MODS Endotype (M1 relative to M2) | 1.29 | 0.68 | 0.06 |
| Adjuvant steroids (Yes vs. No) | −0.26 | 0.78 | 0.75 |
| Interaction between MODS endotype and receipt of steroids. | 0.79 | 1.35 | 0.56 |
| Day 7 Multiple organ dysfunction | | | |
| Constant | −0.69 | 0.35 | 0.05 |
| MODS Endotype (M1 relative to M2) | 1.47 | 0.61 | 0.02 |
| Adjuvant steroids (Yes vs. No) | 0.77 | 0.52 | 0.14 |
| Interaction between MODS endotype and receipt of steroids. | 12 | 268 | 0.97 |

TABLE 28
Reactome pathway of the 50 genes used to identify pediatric sepsis subclasses
based on signatures correlated with MODS trajectories, with FDR < 0.05.

| | Entities | Entities | Entities | Entities p | |
|---|---|---|---|---|---|
| Pathway name | found | total | ratio | value | Entities FDR |
| Neutrophil degranulation | 13 | 480 | 0.0317 | 9.40E−08 | 0.00003 |
| Immune system | 29 | 2698 | 0.17818 | 3.62E−07 | 0.00005 |
| Innate immune system | 19 | 1345 | 0.088826 | 1.78E−06 | 0.00017 |
| MHC class ii antigen presentation | 6 | 149 | 0.00984 | 4.18E−05 | 0.00267 |
| Trafficking and processing of endosomal | 3 | 16 | 0.001057 | 4.68E−05 | 0.00267 |
| TLR1 | | | | | |
| RUNX1 regulates genes involved in | 4 | 78 | 0.005151 | 3.50E−04 | 0.01678 |
| megakaryocyte differentiation and platelet | | | | | |
| function | | | | | |
| Fibronectin matrix formation | 2 | 7 | 4.62E−04 | 4.23E−04 | 0.01733 |
| Transcriptional regulation by RUNX1 | 6 | 261 | 0.017237 | 8.36E−04 | 0.0301 |
| RUNX1 regulates transcription of genes | 2 | 11 | 7.26E−04 | 0.0010325 | 0.03304 |
| involved in differentiation of keratinocytes | | | | | |
| Metal sequestration by antimicrobial proteins | 2 | 13 | 8.59E−04 | 0.00143426 | 0.04016 |

### Example 10

### Comparison of Original and Newly Derived Endotypes of Pediatric Sepsis

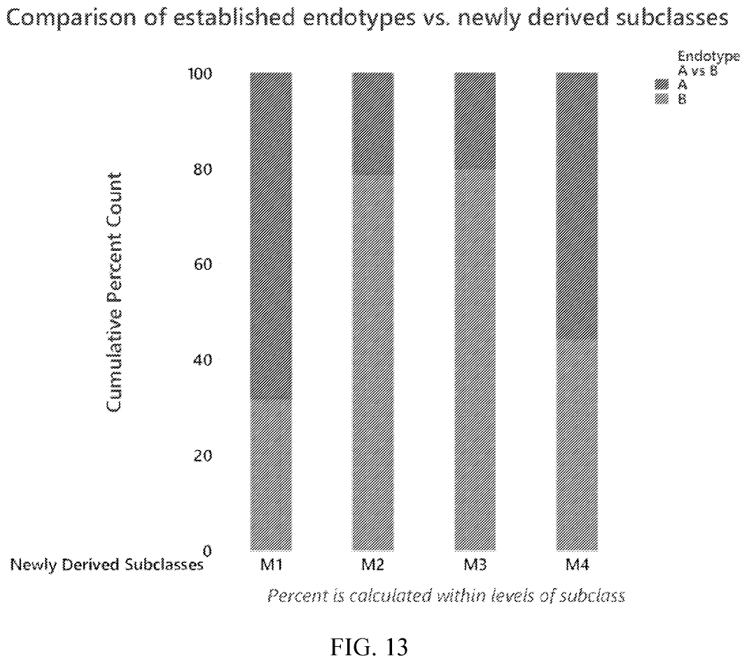

FIG. 13 shows the comparison between previously established endotypes and newly derived patient subclasses among children with septic shock. Endotype A patients were overrepresented among M1 (15/22, 69.2%) and M4 subclasses (5/9, 55.6%); Endotype B were overrepresented among M2 (44/52, 78.9%) and M3 (24/30, 80%) subclasses (x2, p<0.001).

### Example 11

### Estimation of MODS Risk and Identification of Novel Endotypes Via Machine Learning Results in Meaningful Differences in Clinical Outcomes

The above-described examples present a gene expression signature associated with a persistent MODS trajectory and persistent individual organ dysfunctions from whole blood of children with septic shock. Further, deploying supervised machine learning allowed for the discovery of a parsimonious set of 20 genes (namely, RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8) and a fixed classifier model which can be used to reliably estimate risk of MODS across validation datasets, including children and adults with different inciting causes for organ dysfunctions, and can enable identification of novel patient subclasses with meaningful differences in clinical outcomes. This model also demonstrates greater reproducibility in accurately identifying patients with persistent MODS, relative to a gene-set previously established to predict sepsis mortality.

Gene-expression studies among pediatric patients with sepsis explicitly focused on MODS as an outcome have thus far been limited by patient sample size and case-control study design. Snyder et al. profiled 32 children with pediatric sepsis of whom 19 had an immunoanalysis phenotype of MODS and identified 2,303 DEGs, a majority of which were related to innate and adaptive immune systems [19]. Rama Shankar et al. profiled a total of 27 pediatric septic shock patients and identified 30 DEGs when comparing those receiving extracorporeal membrane oxygenation (ECMO) life support (n=6) relative to those with MODS not receiving ECMO; a majority of genes belonged to the histone family [17]. In comparison, the present examples used microarray data from a large prospective cohort of children with septic shock and identified 568 DEGs among patients with persistent MODS relative to those with resolving or no MODS.

The results of biological pathway analyses of gene-expression profiles associated with a persistent MODS trajectory demonstrated an overactive innate immune response with a key role for neutrophil degranulation. The gene-expression signature identified as described herein is very similar to prolonged MODS signature associated with pediatric patients with critical influenza, assessed by quantitative measurement of mRNA transcripts using a Nanostring platform [20]. In addition, they bear striking similarities with adults with a reactive or hyper-inflammatory high-risk phenotype of acute respiratory distress syndrome (ARDS) [28]. Among the top differentially expressed genes in the dataset, several, including RETN, LCN2, IL1R2, CEACAM8, and MPO, were all identified to contribute to neutrophil subset specific responses and emergency granulopoiesis in multi-omic single cell analyses of immune cell subsets among septic patients by Kwok et al. [29]. Taken together with the reproducibility of the predictive capabilities of this ML model to estimate risk of MODS across varying causes of organ dysfunctions including sepsis and trauma in the current study, it is evident that this model is highly biologically relevant and generalizable across critical illness syndromes.

Results of CIBERSORT analyses revealed no significant differences in proportion of neutrophil or monocyte abundance between groups. However, an overabundance of M0 macrophages and plasma cells was identified, along with relatively fewer CD8+ T cells, γδ T cells, and memory B cells. This pattern of innate immune expansion with suppression of the adaptive immune arm has been consistently noted in sepsis [12], and recently demonstrated to drive an extreme response endotype among septic patients [29]. Although these data are extrapolations from bulk RNA microarrays, the consistency with single cell datasets strengthen the findings. Targeted modulation of immune cell subpopulations that drive organ dysfunctions can therefore likely be used as a novel therapeutic approach.

The prognostic utility of the gene-expression classifier among critically ill patients with persistent organ failures has been demonstrated as described herein. This approach has several strengths, including use of supervised ML to identify a limited set of genes consistently associated with the outcome of interest, model optimization in a separate validation set, and demonstration of reproducibility across 2 independent test sets. The 20 gene-classifier, including RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8, more reliably predicted risk of persistent MODS in validation and test sets compared to an established 51 gene-set predictive of sepsis mortality, optimized through similar approaches.

These findings indicate that gene-expression signatures predictive of sepsis mortality may not be sufficient to consistently identify survivors with persistent organ failures nor generalizable across various phenotypes of organ failures. Future studies can prospectively validate these findings and leverage the gene-expression signature of persistent MODS to identify biologically relevant subclasses or endotypes, which may hold potential to demonstrate heterogeneity of treatment effect with modulators of the innate immune response among patients [30-32].

Subsequently, using ML and propensity matching, a stable set of features was identified to reliably estimate risk of MODS in the derivation cohort. Then, using a fixed set of the top 20 genes (RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8) and classifier model demonstrated consistent performance across validation cohorts with substantial clinical heterogeneity. The ability to reliably identify the MODS signature across cohorts with differences in host developmental age and inciting cause of MODS is likely a reflection of shared biological pathways [38].

By eliminating redundancies between the shared MODS signature and those of individual organ dysfunctions, this study demonstrates the ability to identify genes correlated with persistent cardiovascular, lung, and kidney dysfunction. Of considerable significance, CCR1 (C-C motif chemokine receptor 1) [48], GPR34 (G-protein coupled receptor 34) [49], and PDSS1 (Decaprenyl diphosphate synthase subunit 1 that codes for Co-enzyme Q10) [50], have been previously correlated with cardiovascular inflammation or dysfunction; HSPB1 (Heat shock protein family B, small member) [51], LILRA3 (Leukocyte immunoglobulin-like receptor A3)[52], and GSTO1 (Glutathione transferase omega 1) [53] have been previously correlated with respiratory inflammation or dysfunction; CXCL5 (C-X-C motif chemokine ligand 5) [54], CKLF (Colon Krüppel-like factor) [55], and NRGN [56] have been correlated with renal inflammation or dysfunction. Differential expression of such genes among immune cellular subsets, with consequent flux in their respective serum protein concentrations, can result in patterns of organ dysfunction that are commensurate with organ-specific tissue receptor expression. This approach can facilitate identification of novel organ-specific molecular targets for subsequent mechanistic studies and development of targeted therapeutics.

Four subclasses of pediatric sepsis were identified based on 50 top features selected through ML. Of these, M1 and M2 endotypes were enriched for patients with MODS progression and had significantly worse clinical outcomes. Of importance, the newly derived patient subclasses did not show the differential response to corticosteroids, as has been detailed previously based on the endotyping schema used by Wong and colleagues [15,39,40].

Biological pathway analyses suggested a differential role for nuclear transcription factor RUNX1 among patient subclasses. RUNX1 has been implicated in sepsis [57] with a major role in inflammatory tumor necrosis factor (TNF) production. Loss of RUNX1 is thought to activate a transcriptional signature that primes neutrophils to hyper respond to toll-like 4 receptor (TLR4) stimulation [58], as evidenced based on differential expression of key neutrophil genes among those with the M1 vs. M2 endotype. In addition, RUNX1 binds the promoter of the CSF2 gene that encodes granulocyte monocyte colony stimulating factor (GM-CSF) [59]. The latter is of considerable interest because exogenous GM-CSF has shown promise among patients with an immunoparalysis MODS phenotype [3,60] and is currently under investigation as a potential therapeutic agent (GRACE study, NCT03769844). Patients of M2 endotype can overlap with such a phenotype and thus potentially benefit from GM-CSF or RUNX1 modulation.

Comparison of established pediatric sepsis endotypes A and B with the newly derived subclasses described herein demonstrated that patients with endotype A were overrepresented among M1 and M4 subclasses, and those with endotype B were overrepresented among M2 and M3 subclasses. These data demonstrate the existence of complex sub-endotypes among septic patients, as have been described in asthma [61,62], wherein the same individual can both demonstrate differential response to corticosteroids, and simultaneously have a biological predilection to respond to another therapeutic agent. In other words, although endotype A patients are predisposed to respond poorly to steroids, they can have differential regulation of biological pathways that make them amenable to an alternative therapeutic agent given their significant overlap with the M1 endotype. Conversely, a subset of endotype A patients who overlap with the M4 endotype can require no additional therapies given that their risk for mortality or organ dysfunctions may be relatively low. This approach can lead to the development of a tiered-endotyping strategy to identify patients most likely to benefit from an array of targeted therapies and in the future aide clinical decision making at the bedside.

Future cohorts enriched for children with MODS and its related clinical subphenotypes [7] can shed further light on the underlying biology, as can whole blood transcriptional signatures correlated with individual organ dysfunctions [63] via identification of corresponding epithelial, parenchymal, or endothelial molecular targets within organ-specific tissue beds. Temporal transcriptomic shifts and endotyping switching, can also be studied, given that these processes are well documented between day 1 and 3 in pediatric septic shock [64,34,33]. Single-cell RNA sequencing, as has been previously demonstrated [65], can further delineate cell-specific molecular targets correlated with development of organ dysfunctions. Future studies that integrate transcriptomic and epigenomic shifts in sepsis can enable discovery of novel epigenetic therapies that correspond with patient endotypes, given that transcription is tightly regulated by epigenomic changes, which are known to themselves modulate development of organ dysfunctions [66].

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the disclosure. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Weiss S L, Fitzgerald J C, Pappachan J et al. Global Epidemiology of Pediatric Severe Sepsis: The Sepsis Prevalence, Outcomes, and Therapies Study. Am J Respir Crit Care Med 2015; 191:1147-57.
2. Carlton E F, Donnelly J P, Hensley M K et al. New Medical Device Acquisition During Pediatric Severe Sepsis Hospitalizations. Crit Care Med 2020; 48:725-31.
3. Hall M W, Knatz N L, Vetterly C et al. Immunoparalysis and nosocomial infection in children with multiple organ dysfunction syndrome. Intensive Care Med 2011; 37:525-32.
4. Zimmerman J J, Banks R, Berg R A et al. Critical Illness Factors Associated With Long-Term Mortality and Health Related Quality of Life Morbidity Following Community-Acquired Pediatric Septic Shock. Crit Care Med 2020; 48:319-28.
5. Wiens M O, Bone J N, Kumbakumba E et al. Mortality after hospital discharge among children younger than 5 years admitted with suspected sepsis in Uganda: a prospective, multisite, observational cohort study. The Lancet Child & Adolescent Health 2023; 7:555-66.
6. Carcillo J A, Podd B, Aneja R et al. Pathophysiology of Pediatric Multiple Organ Dysfunction Syndrome. Pediatr Crit Care Med 2017; 18:532-45.
7. Carcillo J A, Halstead E S, Hall M W et al. Three hypothetical inflammation pathobiology phenotypes and pediatric sepsis-induced multiple organ failure outcome. Pediatr Crit Care Med 2017; 18:513-23.
8. Wong H R, Cvijanovich N, Lin R et al. Identification of pediatric septic shock subclasses based on genome-wide expression profiling. BMC Med 2009; 7:34.
9. Davenport E E, Burnham K L, Radhakrishnan J et al. Genomic landscape of the individual host response and outcomes in sepsis: a prospective cohort study. Lancet Respir Med 2016; 4:259-71.
10. Scicluna B P, van Vught L A, Zwinderman A H et al. Classification of patients with sepsis according to blood genomic endotype: a prospective cohort study. Lancet Respir Med 2017; 5:816-26
11. Pelaia T M, Shojaei M, McLean A S. The Role of Transcriptomics in Redefining Critical Illness. Critical Care 2023; 27:89.
12. Sweeney T E, Perumal T M, Henao R et al. A community approach to mortality prediction in sepsis via gene expression analysis. Nat Commun 2018; 9:694.
13. Cano-Gamez E, Burnham K L, Goh C et al. An immune dysfunction score for stratification of patients with acute infection based on whole-blood gene expression. Science Translational Medicine 2022; 14:eabg4433.
14. Sweeney T E, Azad T D, Donato M et al. Unsupervised analysis of transcriptomics in bacterial sepsis across multiple datasets reveals three robust clusters. Crit Care Med 2018; 46:915-25.
15. Wong H R, Cvijanovich N Z, Anas N et al. Developing a clinically feasible personalized medicine approach to pediatric septic shock. Am J Respir Crit Care Med 2015; 191:309-15.
16. Antcliffe D B, Burnham K L, Al-Beidh F et al. Transcriptomic Signatures in Sepsis and a Differential Response to Steroids. From the VANISH Randomized Trial. Am J Respir Crit Care Med 2019; 199:980-6.
17. Shankar R, Leimanis M L, Newbury P A et al. Gene expression signatures identify paediatric patients with multiple organ dysfunction who require advanced life support in the intensive care unit. EBioMedicine 2020; 62:103122.
18. Banerjee S, Mohammed A, Wong H R et al. Machine Learning Identifies Complicated Sepsis Course and Subsequent Mortality Based on 20 Genes in Peripheral Blood Immune Cells at 24 H Post-ICU Admission. Front Immunol 2021; 12:592303.
19. Snyder A, Jedreski K, Fitch J et al. Transcriptomic Profiles in Children With Septic Shock With or Without Immunoparalysis. Front Immunol 2021; 12:733834.
20. Novak T, Crawford J C, Hahn G et al. Transcriptomic profiles of multiple organ dysfunction syndrome phenotypes in pediatric critical influenza. Front Immunol 2023; 14:1220028.
21. Atreya M R, Cvijanovich N Z, Fitzgerald J C et al. Integrated PERSEVERE and endothelial biomarker risk model predicts death and persistent MODS in pediatric septic shock: a secondary analysis of a prospective observational study. Critical Care 2022; 26:210.
22. Sweeney T E, Shidham A, Wong H R et al. A comprehensive time-course-based multicohort analysis of sepsis and sterile inflammation reveals a robust diagnostic gene set. Sci Transl Med 2015; 7:287ra71.
23. Ritchie M E, Phipson B, Wu D et al. limma powers differential expression analyses for RNA-sequencing and microarray studies. Nucleic Acids Res 2015; 43:e47.
24. Yu G, Wang L-G, Han Y et al. clusterProfiler: an R package for comparing biological themes among gene clusters. OMICS 2012; 16:284-7.
25. Newman A M, Liu C L, Green M R et al. Robust enumeration of cell subsets from tissue expression profiles. Nat Methods 2015; 12:453-7.
26. Cabrera C P, Manson J, Shepherd J M et al. Signatures of inflammation and impending multiple organ dysfunction in the hyperacute phase of trauma: A prospective cohort study. PLoS Med 2017; 14:e1002352.
27. Chicco D, Jurman G. An Invitation to Greater Use of Matthews Correlation Coefficient in Robotics and Artificial Intelligence. Frontiers in Robotics and AI 2022; 9.
28. Bos L D J, Scicluna B P, Ong D S Y et al. Understanding Heterogeneity in Biologic Phenotypes of Acute Respiratory Distress Syndrome by Leukocyte Expression Profiles. Am J Respir Crit Care Med 2019; 200:42-50.
29. Kwok A J, Allcock A, Ferreira R C et al. Neutrophils and emergency granulopoiesis drive immune suppression and an extreme response endotype during sepsis. Nat Immunol 2023; 24:767-79.
30. Bo L, Wang F, Zhu J et al. Granulocyte-colony stimulating factor (G-CSF) and granulocyte-macrophage colony stimulating factor (GM-CSF) for sepsis: a meta-analysis. Critical Care 2011; 15:R58.
31. Interleukin-6 Receptor Antagonists in Critically Ill Patients with Covid-19. New England Journal of Medicine 2021; 384:1491-502.
32. Karakike E, Scicluna B P, Roumpoutsou M et al. Effect of intravenous clarithromycin in patients with sepsis, respiratory and multiple organ dysfunction syndrome: a randomized clinical trial. Crit Care 2022; 26:183.
33. Wong H R, Cvijanovich N Z, Anas N et al. Endotype Transitions During the Acute Phase of Pediatric Septic Shock Reflect Changing Risk and Treatment Response. Crit Care Med 2018; 46:e242-9.
34. Maslove D M, Wong H R. Gene expression profiling in sepsis: timing, tissue, and translational considerations. Trends Mol Med 2014; 20:204-13.
35. Marshall J C. Why have clinical trials in sepsis failed?Trends Mol Med 2014; 20:195-203.
36. Atreya M R, Wong H R. Precision medicine in pediatric sepsis. Curr Opin Pediatr 2019; 31:322-7.
37. Stanski N L, Wong H R. Prognostic and predictive enrichment in sepsis. Nat Rev Nephrol 2020; 16:20-31.
38. Wong H R, Cvijanovich N Z, Allen G L et al. VALIDATION OF A GENE EXPRESSION-BASED SUBCLASSIFICATION STRATEGY FOR PEDIATRIC SEPTIC SHOCK. Crit Care Med 2011; 39:2511-7.
39. Wong H R, Atkinson S J, Cvijanovich N Z et al. Combining Prognostic and Predictive Enrichment Strategies to Identify Children With Septic Shock Responsive to Corticosteroids. Crit Care Med 2016; 44:e1000-1003.
40. Wong H R, Hart K W, Lindsell C J et al. External Corroboration That Corticosteroids May Be Harmful to Septic Shock Endotype A Patients. Crit Care Med 2021; 49:e98-101.
41. Fabregat A, Jupe S, Matthews L et al. The Reactome Pathway Knowledgebase. Nucleic Acids Res 2018; 46:D649-55.
42. Kalousis A, Prados J, Hilario M. Stability of feature selection algorithms: a study on high-dimensional spaces. Knowl Inf Syst 2007; 12:95-116.
43. Almansa R, Tamayo E, Heredia M et al. Transcriptomic evidence of impaired immunoglobulin G production in fatal septic shock. Journal of Critical Care 2014; 29:307-9.
44. Almansa R, Heredia-Rodriguez M, Gomez-Sanchez E et al. Transcriptomic correlates of organ failure extent in sepsis. Journal of Infection 2015; 70:445-56.
45. Erickson B J, Kitamura F. Magician's Corner: 9. Performance Metrics for Machine Learning Models. Radiol Artif Intell 2021; 3:e200126.
46. Pollack M M, Patel K M, Ruttimann U E. The Pediatric Risk of Mortality III—Acute Physiology Score (PRISM III-APS): a method of assessing physiologic instability for pediatric intensive care unit patients. J Pediatr 1997; 131:575-81.
47. Weiss S L, Carcillo J A, Leclerc F et al. Refining the Pediatric Multiple Organ Dysfunction Syndrome. Pediatrics 2022; 149:S13-22.
48. Frangogiannis N G, Entman M L. Targeting the Chemokines in Myocardial Inflammation. Circulation 2004; 110:1341-2.
49. Liebscher I, Muller U, Teupser D et al. Altered Immune Response in Mice Deficient for the G Protein-coupled Receptor GPR34. J Biol Chem 2011; 286:2101-10.
50. Brea-Calvo G, Haack T B, Karall D et al. COQ4 Mutations Cause a Broad Spectrum of Mitochondrial Disorders Associated with CoQ10 Deficiency. Am J Hum Genet 2015; 96:309-17.
51. Breed E R, Hilliard C A, Yoseph B et al. The small heat shock protein HSPB1 protects mice from sepsis. Sci Rep 2018; 8:12493.
52. Lewis Marffy A L, McCarthy A J. Leukocyte Immunoglobulin-Like Receptors (LILRs) on Human Neutrophils: Modulators of Infection and Immunity. Frontiers in Immunology 2020; 11.
53. Piaggi S, Marchi E, Carnicelli V et al. Airways glutathione S-transferase omega-1 and its A140D polymorphism are associated with severity of inflammation and respiratory dysfunction in cystic fibrosis. J Cyst Fibros 2021; 20:1053-61.
54. Disteldorf E M, Krebs C F, Paust H-J et al. CXCL5 Drives Neutrophil Recruitment in TH17-Mediated G N. J Am Soc Nephrol 2015; 26:55-66.
55. Li J, Liu L, Zhou W-Q et al. Roles of Krüppel-like factor 5 in kidney disease. Journal of Cellular and Molecular Medicine 2021; 25:2342-55.
56. Dumas S J, Meta E, Borri M et al. Phenotypic diversity and metabolic specialization of renal endothelial cells. Nat Rev Nephrol 2021; 17:441-64.
57. Luo M-C, Zhou S-Y, Feng D-Y et al. Runt-related Transcription Factor 1 (RUNX1) Binds to p50 in Macrophages and Enhances TLR4-triggered Inflammation and Septic Shock. Journal of Biological Chemistry 2016; 291:22011-20.
58. Bellissimo D C, Chen C, Zhu Q et al. Runxl negatively regulates inflammatory cytokine production by neutrophils in response to Toll-like receptor signaling. Blood Advances 2020; 4:1145-58.
59. Oakford P C, James S R, Qadi A et al. Transcriptional and epigenetic regulation of the GM-CSF promoter by RUNX1. Leuk Res 2010; 34:1203-13.
60. Mathias B, Szpila B E, Moore F A et al. A Review of GM-CSF Therapy in Sepsis. Medicine (Baltimore) 2015; 94:e2044.
61. Agache I, Sugita K, Morita H et al. The Complex Type 2 Endotype in Allergy and Asthma: From Laboratory to Bedside. Curr Allergy Asthma Rep 2015; 15:29.
62. Agache I, Akdis C A. Precision medicine and phenotypes, endotypes, genotypes, regiotypes, and theratypes of allergic diseases. J Clin Invest 129:1493-503.
63. Wong H R, Marshall J C. Leveraging Transcriptomics to Disentangle Sepsis Heterogeneity. Am J Respir Crit Care Med 2017; 196:258-60.
64. Kwan A, Hubank M, Rashid A et al. Transcriptional Instability during Evolving Sepsis May Limit Biomarker Based Risk Stratification. PLoS One 2013; 8:e60501.
65. Reyes M, Filbin M R, Bhattacharyya R P et al. An immune cell signature of bacterial sepsis. Nat Med 2020; 26:333-40.
66. Falcio-Holanda R B, Brunialti MKC, Jasiulionis M G et al. Epigenetic Regulation in Sepsis, Role in Pathophysiology and Therapeutic Perspective. Frontiers in Medicine 2021; 8.
67. Marshall J C, Deutschman C S. The Multiple Organ Dysfunction Syndrome: Syndrome, Metaphor, and Unsolved Clinical Challenge. Crit Care Med 2021; 49:1402-13.
68. Wong H R, Cvijanovich N, Allen G L et al. Genomic expression profiling across the pediatric systemic inflammatory response syndrome, sepsis, and septic shock spectrum. Crit Care Med 2009; 37:1558-66
69. Geurts P, Ernst D, Wehenkel L. Extremely randomized trees. Mach Learn 2006; 63:3-42.

1. A method of classifying a patient with septic shock as high risk of persistent multiple organ dysfunction syndrome (MODS) trajectory and/or mortality or other than high risk of persistent MODS trajectory and/or mortality, the method comprising:
obtaining a sample from a pediatric patient with septic shock at a first time point;
analyzing the sample to determine gene expression levels of two or more biomarkers selected from the group consisting of: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8;
determining whether the gene expression levels of each of the at least two biomarkers are differentially expressed normalized gene expression levels; and
classifying the patient as high risk of persistent MODS trajectory and/or mortality, or other than high risk of persistent MODS trajectory and/or mortality, based on the determination of whether the expression levels of each of the at least two biomarkers are differentially expressed normalized gene expression levels.

2. The method of claim 1, wherein a classification of high risk of persistent MODS trajectory and/or mortality comprises a differentially expressed normalized expression level of 3, 4, 5, 6, 7, 8, 9, 10, 11, 12, 13, 14, 15, 16, 17, 18, 19, 20, or more biomarkers selected from the group consisting of: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8.

3. The method of claim 1, wherein a classification of high risk of persistent MODS trajectory and/or mortality comprises a non-differentially expressed normalized gene expression level of RUNX1.

4. The method of claim 1, wherein biomarker expression levels are determined by quantification of serum biomarker concentrations, and/or wherein gene expression levels are determined by concentrations and/or by the cycle threshold (CT) values.

5. (canceled)

6. The method of claim 1, wherein determining whether the gene expression levels of each of the at least two biomarkers are differentially expressed normalized gene expression levels comprises comparing the gene expression levels to respective gene expression levels from a normal, healthy subject.

7. The method of a claim 1, wherein the patient is classified as high risk of persistent MODS trajectory and/or mortality, or other than persistent MODS trajectory and/or mortality, when the expression levels of each of the at least two biomarkers are differentially expressed normalized gene expression levels as compared to respective gene expression levels from a normal, healthy subject.

8. The method of claim 1, wherein a classification of high risk of persistent MODS trajectory and/or mortality comprises a differentially expressed normalized expression level of 3, 4, 5, 6, 7, 8, 9, 10, 11, 12, 13, 14, 15, 16, 17, 18, 19, or 20 biomarkers selected from the group consisting of: RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8; or wherein a classification of high risk of persistent MODS trajectory and/or mortality comprises a differentially expressed normalized expression level of 20 biomarkers comprising: RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8; or wherein a classification of high risk of persistent MODS trajectory and/or mortality comprises a differentially expressed normalized gene expression level of 3, 4, 5, 6, 7, 8, 9, or 10 biomarkers selected from the group consisting of: RETN, ADAMTS3, LDHA, LCN2, IL1R2, DDIT4, CEACAM8, MERTK, MPO, and ARL4A; or wherein a classification of high risk of persistent MODS trajectory and/or mortality comprises differentially expressed normalized expression levels of all biomarkers selected from the group consisting of: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8.

9. (canceled)

10. (canceled)

11. (canceled)

12. The method of claim 1, wherein a classification other than high risk comprises a classification of low risk or intermediate risk.

13. The method of claim 1, wherein MODS comprises cardiovascular, respiratory, renal, hepatic, hematologic, and/or neurologic dysfunction, and/or dysfunction in one or more organs selected from heart, lungs, kidneys, liver, blood, and brain.

14. The method of claim 13, wherein MODS comprises cardiovascular dysfunction.

15. (canceled)

16. The method of claim 1, wherein high risk of persistent MODS trajectory and/or mortality by day 7 of septic shock or other than high risk of persistent MODS trajectory and/or mortality by day 7 of septic shock is determined.

17. The method of claim 1, wherein the classification is combined with one or more patient demographic data and/or clinical characteristics and/or results from other tests or indicia of septic shock and/or one or more additional biomarkers, and/or one or more additional population-based risk scores.

18. The method of claim 17, wherein the one or more additional biomarkers is selected from the group consisting of: C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-2 (Angpt-2), Tyrosine kinase with immunoglobulin-like loops and epidermal growth factor homology domains-2 (Tie-2), Vascular cell adhesion molecule-1 (VCAM-1), P-selectin, E-selectin, and Platelet and endothelial cell adhesion molecule-1 (PECAM-1); and/or wherein the patient demographic data and/or clinical characteristics and/or results from other tests or indicia of septic shock comprise at least one selected from the group consisting of: the septic shock causative organism, the presence or absence or chronic disease, and/or the age, gender, race, and/or co-morbidities of the patient.

19. (canceled)

20. (canceled)

21. (canceled)

22. The method of claim 1, wherein the sample is obtained within the first hour of presentation with septic shock; or wherein the sample is obtained within the first 24 hours of presentation with the septic shock.

23. (canceled)

24. The method of claim 1, further comprising administering a treatment comprising one or more high risk therapy to a patient that is classified as high risk of persistent MODS trajectory and/or mortality, or administering a treatment excluding a high risk therapy to a patient that is not high risk of persistent MODS trajectory and/or mortality, or to provide a method of treating a pediatric patient with septic shock.

25. The method of claim 24, wherein the one or more high risk therapy comprises at least one selected from the group consisting of: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies.

26. The method of claim 25, wherein the biological and/or immune enhancing therapy comprises administration of GM-CSF, Interleukin-1 receptor antagonist, Interleukin-7, RUNX1 modulation, and/or anti-PD-1.

27. The method of claim 1, wherein the patient is enrolled in a clinical trial; or wherein the patient is enrolled in a clinical trial and is classified as high risk.

28. (canceled)

29. (canceled)

30. (canceled)

31. (canceled)

32. The method of claim 24, further comprising:
obtaining a second sample from the treated patient at a second time point;
analyzing the second sample to determine gene expression levels of two or more biomarkers selected from the group consisting of: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8;
determining whether the gene expression levels of each of the at least two biomarkers are differentially expressed normalized gene expression levels;
classifying the patient as high risk of persistent MODS trajectory and/or mortality, or other than high risk of persistent MODS trajectory and/or mortality, based on the determination of whether the expression levels of each of the biomarkers are differentially expressed normalized gene expression levels; and
maintaining the treatment being administered if the patient's high risk classification has not changed, or changing the treatment being administered if the patient's high risk classification has changed.

33. The method of claim 32, wherein the second time point is at least 18 hours after the first time point; or wherein the second time point is in the range of 24 to 96 hours, or longer, after the first time point; or wherein the second time point is about 1 day, 2 days, 3 days, or longer, after the first time point; or wherein the first time point is at day 1, wherein day 1 is within 24 hours of a septic shock diagnosis, and the second time point is at day 3.

34. (canceled)

35. (canceled)

36. (canceled)

37. (canceled)

38. The method of claim 32, wherein a patient classified as high risk after the second time point is administered one or more high risk therapy; or wherein a patient not classified as high risk after the second time point is administered a treatment excluding a high risk therapy; and/or wherein the patient classified as high risk and administered one or more high risk therapy after the first time point is not classified as high risk after the second time point.

39. (canceled)

40. (canceled)

41. (canceled)

42. (canceled)

43. The method of claim 1, further comprising receiving a sample dataset, wherein the sample dataset comprises mRNA from a subject having MODS or from a MODS cohort, and analyzing the sample dataset by a machine learning model to identify two or more genes associated with a persistent MODS trajectory and/or mortality.

44. (canceled)

45. A diagnostic kit, test, or array comprising a reporter hybridization probe, and a capture hybridization probe specific for each of two or more mRNA, DNA, or protein biomarkers selected from the group consisting of: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8; and/or wherein the biomarkers comprise three or more selected from the group consisting of: GGH, NUSAP1, ZBTB16, HLA-DMB, IL1RAP, ANLN, MTMR11, PRTN3, CDKN3, HLA-DPA1, LTF, PNPLA6, H1-2, SLC51A, FCGR2B, SLC46A2, GPI, TLR7, CEP55, ARL4A, MPO, MERTK, CEACAM8, DDIT4, IL1R2, LCN2, LDHA, ADAMTS3, RETN, FCER1A, CEACAM6, MAP3K7CL, OLFM4, BLM, NFE2, MAFF, CTSL, CD24, PRC1, SLCO4A1, IFI44L, ORM1, DAAM2, PRG2, TAGAP, CEACAM1, FGFBP2, CENPW, MS4A4A, RUNX1, and MMP8; and/or wherein the biomarkers comprise RETN, ADAMTS3, LDHA, LCN2, IL1R2, DDIT4, CEACAM8, MERTK, MPO, ARL4A, CDKN3, PRTN3, MTMR11, ANLN, IL1RAP, HLA-DMB, ZBTB16, NUSAP1, GGH, and MMP8; and/or wherein the biomarkers comprise RETN, ADAMTS3, LDHA, LCN2, IL1R2, DDIT4, CEACAM8, MERTK, MPO, and ARL4A.

46. (canceled)

47. (canceled)

48. (canceled)

49. (canceled)

50. (canceled)

51. (canceled)

52. (canceled)

53. (canceled)

54. (canceled)

55. A method of classifying a patient with septic shock as high risk of cardiovascular, respiratory, or renal dysfunction or other than high risk of cardiovascular, respiratory, or renal dysfunction, the method comprising:
obtaining a sample from a pediatric patient with septic shock at a first time point;
analyzing the sample to determine gene expression levels of two or more biomarkers selected from genes listed in Tables 13-24;
determining whether the gene expression levels of each of the at least two biomarkers are differentially expressed normalized gene expression levels; and
classifying the patient as high risk of cardiovascular, respiratory, or renal dysfunction, or other than high risk of cardiovascular, respiratory, or renal dysfunction, based on the determination of whether the expression levels of each of the at least two biomarkers are differentially expressed normalized gene expression levels.

56. The method of claim 55, wherein determining whether the gene expression levels of each of the at least two biomarkers are differentially expressed normalized gene expression levels comprises comparing the gene expression levels to respective gene expression levels from a normal, healthy subject; and/or wherein the patient is classified as high risk of cardiovascular, respiratory, or renal dysfunction, or other than high risk of cardiovascular, respiratory, or renal dysfunction, when the expression levels of each of the at least two biomarkers are differentially expressed normalized gene expression levels as compared to respective gene expression levels from a normal, healthy subject.

57. (canceled)
